(19) United States
(12) Patent Application Publication
(10) Pub. No.: US 2020/0282075 A1
(43) Pub. Date: Sep. 10, 2020

(54) ALBUMIN-MODIFIED NANOPARTICLES CARRYING A TARGETING LIGAND

(71) Applicant: AbbVie Deutschland GmbH & Co. KG, Wiesbaden (DE)

(72) Inventors: Lance KALETA, Ludwigshafen (DE); Axel MEYER, Ludwigshafen (DE); Christian RIED, Ludwigshafen (DE); Michael ROHE, Ludwigshafen (DE); Kathrin SCHÄKER-THEOBALD, Ludwigshafen (DE); Sonja TALMON, Ludwigshafen (DE); Christopher UNTUCHT, Ludwigshafen (DE); Tina ZIMMERMANN, Ludwigshafen (DE)

(21) Appl. No.: 16/645,216

(22) PCT Filed: Sep. 6, 2018
(86) PCT No.: PCT/EP2018/073975

§ 371 Date: Mar. 6, 2020

Related U.S. Application Data
(60) Provisional application No. 62/555,254, filed on Sep. 7, 2017.

(30) Foreign Application Priority Data
Sep. 7, 2017 (EP) 17189996.6

(51) Int. Cl. A61K 47/69; A61K 38/40; A61K 47/62; A61K 49/00

(52) U.S. Cl. CPC A61K 47/6929; A61K 38/40; A61K 9/5123; A61K 49/0058; A61K 49/0093; A61K 47/62

(57) ABSTRACT

The present invention relates to cargo substance-loaded, albumin-modified nanoparticles comprising a targeting ligand, to a method for producing such nanoparticles, to nanoparticles obtainable by said method, to a pharmaceutical composition containing a plurality of such nanoparticles and to the medical use of such nanoparticles.

### FIELD OF THE INVENTION

The present invention relates to cargo substance-loaded, albumin-modified nanoparticles comprising a targeting ligand, to a method for producing such nanoparticles, to nanoparticles obtainable by said method, to a pharmaceutical composition containing a plurality of such nanoparticles and to the medical use of such nanoparticles.

### BACKGROUND OF THE INVENTION

The term “nanoparticles” is generally used to designate particles having a diameter in the nanometer range. Nanoparticles include particles of different structure, such as nanocapsules and matrix particles.

Nanoparticles have been studied as drug delivery systems and in particular as systems for targeting drugs to specific sites of action within the patient for several years. They have the potential to become the leading vehicle for disease diagnosis and therapy. Nanoparticles offer an improved solubility, enhanced bioavailability, increased exposure of the target tissue to the drug and lower the dose required for the desired effect. At the same time, however, the small size, which is associated with a very large surface-to-volume ratio, also leads to some undesired effects. For instance, it has been observed that once the nanoparticles enter a biological medium, such as blood, they are immediately coated by proteins, forming a so-called “protein corona”. This protein corona not only enhances the particles' size, but, more importantly, masks the original, desired properties of the initial nanoparticle, since this corona appears to be what is actually detected by the cells and the organs and thus defines the biological identity of the particle. This can alter the biological responses to the particle completely. For instance, among the proteins which can bind to the nanoparticles is a specific class called opsonins (e.g. immunoglobulin IgG and complement), and as their name indicates, they play an important role in opsonization. Absorption of opsonins onto the nanoparticle surface promotes phagocytosis of the nanoparticles, thus leading to their rapid clearance from blood circulation after intravenous application. Also the enhanced size of the corona-surrounded nanoparticle is a trigger for phagocytosis. Additionally, the conformation and function of certain corona proteins is altered and results in toxicity. Nanoparticles which absorb proteins in an uncontrolled manner on their surface will thus have only limited use as nanomedicinal products, if at all.

The protein corona problem has been known for some years. One approach to solve this problem is to purposefully form a predetermined protein corona, mostly an albumin corona, around the nanoparticles.

Q. Peng et al. report in Biomaterials 2013, 34, 8521-8530 and in Nanomedicine (Lond.) 2015, 10(2), 205-214 the formation of an albumin corona as a protective coating for a nanoparticle-based drug delivery system. Poly-3-hydroxybutyrate-co-3-hydroxyhexanoate nanoparticles are coated with bovine serum albumin (BSA) by incubation at 4° C., 37° C. or room temperature. The thusly coated particles showed reduced absorption of other plasma proteins from the blood, a reduced clearance rate from blood circulation and a reduced cytotoxicity.

M. Schäffler et al. report in Biomaterials 2014, 35, 3455-3466 on the formation of human serum albumin-coated gold nanoparticles and their potential utility as tool for organ targeting.

S-M. Yu et al. describe in Acta Biomaterialia 2016, 43, 348-357 the purposeful preformation of a protein corona on superparamagnetic iron oxide nanoparticles.

L. K. Müller et al. describe in RCS Advances 2016, 6, 96495-96509 the use of various fractions of human blood plasma for preparing a preformed protein corona for polystyrene or functionalized polystyrene (functionalized with COOH groups from copolymerization of styrene with acrylic acid or functionalized with NH₂ groups from copolymerization of styrene with 2-aminoethyl methacrylate). Cellular uptake of nanoparticles with and without preformed protein corona was investigated using a macrophage-like cell line. Non-functionalized and amino-functionalized polystyrene nanoparticles with preformed protein corona of specific fractions showed a strongly enhanced cellular uptake as compared to naked nanoparticles, while other fractions showed the opposite effect, i.e. a decrease in cellular uptake. In carboxyl-functionalized polystyrene nanoparticles with preformed protein corona of the latter fractions, no effect was observed as compared to the naked nanoparticles.

An overview over nanomaterials and the predetermined formation of an albumin corona on them is given by J. Mariam et al. in Drug Delivery 2016, 23(8), 2688-2676.

As the studies of L. K. Müller et al. as well as studies of the inventors of the present application show, nanoparticles with a preformed protein corona may solve the problems associated with the uncontrolled formation of a protein corona on nanoparticles once they enter a biological medium, but may have problems with uptake into the targeted cells.

Accordingly, it was the object of the present invention to provide nanoparticles with a good uptake into the targeted cells, which at the same time avoid the problems associated with the uncontrolled formation of a protein corona when introduced into a biological medium, such as blood, and thus show a reduced clearance rate from blood circulation and no or only low undesired cytotoxicity. Moreover, it was a particular aspect of the object of the present invention to provide nanoparticles which are able to cross the blood/brain barrier, and thus can serve as carrier for cargo (e.g., a drug) to be delivered to the brain.

### SUMMARY OF THE INVENTION

The object is achieved by a cargo substance-loaded nanoparticle modified with albumin and a targeting ligand.

Thus, in a first aspect, the invention relates to a cargo substance-loaded nanoparticle modified with albumin and a targeting ligand, comprising
(i) a cargo substance selected from the group consisting of pharmaceutically active agents, cosmetically active agents and nutritional supplements;
(ii) a material which surrounds or embeds the cargo substance;
(iii) an albumin which is covalently directly or indirectly bound to the material (ii); and
(iv) a targeting ligand which is covalently bound to the albumin (iii) via a linker.

The invention moreover relates to a method for producing such nanoparticles, and also to a nanoparticle obtainable by said method.

The invention furthermore relates to a pharmaceutical composition containing a plurality of such nanoparticles.

Another aspect of the invention is the medical use of such nanoparticles; i.e. the nanoparticles of the invention for use as a medicament, and in particular for use in the treatment of CNS disorders; the use of the nanoparticles of the invention for preparing a medicament; the use of the nanoparticles for preparing a medicament for the treatment of disorders, deficiencies or conditions, such as CNS disorders, liver disorders, inflammatory diseases, hyperproliferative diseases, a hypoxia-related pathology and a disease characterized by excessive vascularization; and a method for treating such disorders, deficiencies or conditions, which method comprises administering to a patient in need thereof nanoparticles of the invention.

### DETAILED DESCRIPTION OF THE INVENTION

The term “albumin which is covalently indirectly bound” means that the albumin is bound via linker/linking group to the material (ii). The albumin is bound via a covalent bond to the linker/linking group and the linker/linking group is also bound covalently to the material (ii). “Albumin which is covalently directly bound” means a covalent bond between albumin and material (ii). This is of course only possible if material (ii) has functional groups which can react with the albumin, in particular functional groups which can react with the amino groups of the albumin to give a covalent bond.

### Nanoparticles

Nanoparticles are solid submicron particles having a diameter within the nanometer range (i.e. between several nanometers to several hundred nanometers).

Thus, the nanoparticles of the invention have a mean particle size of at most 1000 nm, e.g. from 1 to 1000 nm or from 10 to 1000 nm or from 20 to 1000 nm; preferably at most 500 nm, e.g. from 1 to 500 nm or from 10 to 500 nm or from 20 to 500 nm; in particular at most 300 nm, e.g. from 1 to 300 nm or from 10 to 300 nm or from 20 to 300 nm; and specifically at most 200 nm, e.g. from 1 to 200 nm or from 10 to 200 nm or from 20 to 200 nm or from 20 to 150 nm or from 50 to 150 nm.

Unless indicated otherwise, the terms “size” and “diameter”, when referring to the nanoparticle of the invention are used interchangeably.

Precisely spoken, the term “diameter” only refers to spherical particles, but in terms of the present invention it is nevertheless also used for less regular geometrical form of the particles and denotes their size as determined by Dynamic Light Scattering.

Size and polydispersity index (PDI) of a nanoparticle preparation can be determined, for example, by Dynamic Light Scattering (DLS, also known as Photon Correlation Spectroscopy or Quasi Elastic Light Scattering) and cumulant analysis according to the International Standard on Dynamic Light Scattering ISO13321 (1996) and ISO22412 (2008) which yields an average diameter (z-average diameter) and an estimate of the width of the distribution (PDI), e.g. using a Zetasizer device (Malvern Instruments, Germany; software version “Nano ZS”). Alternatively, the size of a nanoparticle preparation can be determined, for example, by nanoparticle tracking analysis (NTA) using a NanoSight NS300 device (Malvern Instruments, Germany) which yields a mean particle size as well as D10, D50 and D90 values (wherein D10, D50 and D90 designate diameters, with 10% of the particles having diameters lower than D10, 50% of the particles having diameters lower than D50, and 90% of the particles having diameters lower than D90).

The nanoparticles can protect the cargo substance (i) on the way to the target site (e.g. the target cell) from degradation and/or modification by proteolytic and other enzymes and thus from the loss of their biological (e.g. pharmaceutical) activity. The invention is therefore also particularly useful for encapsulating cargo substances which are susceptible to such enzymatic degradation and/or modification (e.g. polypeptides, peptides).

In the nanoparticles of the invention, the cargo substance (i) is surrounded by or embedded in a material (ii). The material (ii) may form a regular or irregular shell which surrounds the cargo substance (i) or may form a matrix in which the cargo substance (i) is embedded. The cargo substance (i) may be completely or only partly surrounded by or embedded in the material (ii). In particular, the material (ii) will completely surround the cargo substance (i), thereby forming a barrier between this substance and the surrounding medium.

In a preferred embodiment, the nanoparticle is selected from the group consisting of
nanocapsules comprising a shell and a core, where the core comprises the cargo substance and the shell comprises the material (ii) (and to which of course the albumin, the linker and the targeting ligand are bound);
matrix particles containing the material (ii) in form of a matrix in which the cargo substance is embedded (where again the albumin, the linker and the targeting ligand are bound to material (ii)); and
mixed forms thereof.

Nanocapsules are spherical objects which consist of a core and shell, i.e. a wall material surrounding the core. In the nanocapsules of the invention, the core contains the cargo substance (i). The shell comprises the material (ii).

In the core of the nanocapsules of the invention, the cargo substance (i) may be liquid or in the form of a liquid (e.g. aqueous or oily) solution or dispersion, or in an undissolved solid form, such as an amorphous, semi-crystalline or crystalline state, or a mixture thereof.

Matrix particles are amorphous particles which contain the cargo substance (i) embedded in a matrix formed by the material (ii). “Embedded” (also sometimes termed “incorporated”) means that the cargo substance (i) is dispersed within the material (ii).

The nanoparticles can also take a mixed form thereof. A mixed form in this context can be a mixture of nanocapsules and matrix particles. Another example of a mixed form is a nanoparticle in which a core-shell structure containing the cargo substance (i) in the core and material (ii) as a shell is in turn incorporated in a matrix formed by material (ii), or a nanoparticle in which a core-shell structure containing the cargo substance (i) in the core and material (ii) as a shell is in turn incorporated in a matrix formed by material (ii) and the material (ii) additionally contains cargo substance (i) in embedded form. Such mixed core-shell/matrix forms can be distinguished from pure matrix forms when the cargo substance (i) is present in a liquid dispersant, i.e. as solution, suspension or emulsion. In this case, the matrix contains liquid-filled vesicles in which the cargo substance is present (dissolved/suspended/emulsified) in a liquid dispersant.

In a particular embodiment, the nanoparticles are nanocapsules.

In another particular embodiment, the nanoparticles are matrix particles.

In another particular embodiment, the nanoparticles are a mixed form of nanocapsules and matrix particles.

Specifically, the nanoparticle is a mixed form, very specifically a mixed form in which a core-shell structure containing the cargo substance (i) in the core and material (ii) as a shell is in turn incorporated in a matrix formed by material (ii).

### Cargo Substance

The nanoparticle of the invention can contain one or more than one cargo substance (i), e.g. 2, 3 or 4 different cargo substances (i).

The cargo substance (i) is preferably a pharmaceutically active agent. The nature of the pharmaceutically active agent is not limited. However, the cargo substance is expediently a pharmaceutically active agent which is either to be transported to a difficult-to-reach cell, tissue or organ, such as the brain, or which is to be transported selectively to a specific target, such as a cancer cell.

In a specific embodiment, the pharmaceutically active agent is a biopharmaceutical.

“Biopharmaceuticals”, also known as a biologic(al) medical product, biological, or biologic, is any pharmaceutical drug product manufactured in, extracted from, or semi-synthesized from biological sources. Different from totally synthesized pharmaceuticals, they include vaccines, blood, blood components, allergenics, somatic cells, tissues, recombinant therapeutic protein, and living cells used in cell therapy. Biologics can be composed of sugars, proteins, or nucleic acids or complex combinations of these substances, or may be living cells or tissues. Examples for biologics extracted from living systems are whole blood and other blood components, organs and tissue transplants, stem cells for stem cell therapy, antibodies for passive immunization (e.g. to treat a virus infection), human breast milk, fecal microbiota or human reproductive cells. Examples for biologics produced by recombinant DNA are blood factors (Factor VIII and Factor IX), thrombolytic agents (e.g. tissue plasminogen activator), hormones (e.g. insulin, glucagon, growth hormone, gonadotrophins), hematopoietic growth factors (e.g. Erythropoietin, colony stimulating factors), interferons (e.g. Interferons-α, -β, -γ), interleukin-based products (e.g. Interleukin-2), vaccines (e.g. Hepatitis B surface antigen), monoclonal antibodies and others, such as tumor necrosis factor or therapeutic enzymes. Preferably, the biopharmaceuticals are biologics produced by recombinant DNA. In a specific embodiment, the biopharmaceuticals are selected from monoclonal antibodies.

In addition to the cargo compounds, further ingredients can be incorporated (e.g. dissolved or dispersed), for example as described below.

### Material (ii)

The material (ii) which surrounds or embeds the cargo substance can be of any type which is suitable for the use in biological systems, especially in the human organism. Ideally it is non-toxic, biocompatible, non-immunogenic, biodegradable and avoids recognition by the host's defense mechanisms.

Preferably, the material (ii) is selected from the group consisting of lipids, natural polymers, synthetic polymers and carbon nanotubes.

“Lipid” is a broad term for substances of biological origin that are soluble in nonpolar solvents. It comprises a group of naturally occurring molecules that include fats, waxes, sterols, fat-soluble vitamins, monoglycerides, diglycerides, triglycerides, phospholipids, and others. They can be classified into the categories fatty acids, glycerolipids, glycerophospholipids, sphingolipids, glycolipids, polyketides (derived from condensation of ketoacyl subunits), sterol lipids and prenol lipids (derived from condensation of isoprene subunits). In terms of the present invention, the term “lipid” is not restricted to naturally occurring substances, but encompasses synthetically or semisynthetically obtained molecules and also analogues of the naturally occurring molecules.

Preferably, the lipid is selected from such lipids which have a melting point of at least 25° C. More preferably, the lipid is selected from lipids which have a melting point of at least 30° C. In particular, the lipid has a melting point of at least 35° C. If the cargo substance is a substance which is sensitive to elevated temperature and is moreover not expediently exposed to non-polar organic solvents, which is the case for most biopharmaceuticals, the lipid is moreover preferably selected from lipids which have a melting point of at most 55° C. and thus have preferably a melting point of from 25° C. to 55° C., more preferably from 30 to 55° C. and in particular from 35° C. to 55° C. This limitation is due to the fact that substances which are sensitive to elevated temperature and are moreover not expediently exposed to non-polar organic solvents are generally introduced into the lipid by melting the latter and introducing the substance into the melt.

The temperature of the lipid melt, in case of thermically sensitive cargo substances, must of course not exceed a value above which the cargo substance would be negatively affected.

The lipid is preferably selected from the group consisting of triglycerides, diglycerides, monoglycerides, fatty acids, steroids, and waxes.

A triglyceride is an ester derived from glycerol and three fatty acids, where the three fatty acids can be the same or different. Suitable triglycerides are for example caprylic acid triglyceride, trilaurin (synonyms: glycerol trilaurate; glycerin trilaurate; glyceryl trilaurate; trilauroyl glycerol; 1,2,3-propanetriyl tridodecanoate), tripalmitin (synonyms: glycerol tripalmitate; glycerin tripalmitate; glyceryl tripalmitate; palmitic triglyceride; tripalmitoyl glycerol; 1,2,3-propanetriyl trihexadecanoate), trimyristin (synonyms: glycerol trimyristate; glycerin trimyristate; glyceryl trimyristate; trimyristoyl glycerol; 1,2,3-propanetriyl tritetradecanoate) and tristearin (synonyms: glycerol tristearate; glycerin tristearate; glyceryl tristearate; tristearoyl glycerol; 1,2,3-propanetriyl trioctacanoate), and mixed forms, such as laurindipalmitin glyceride, dilaurinpalmitin glyceride, laurindistearin glyceride, dilaurinstearin glyceride and the like.

A diglyceride is an ester derived from glycerol and two fatty acids. There are two possible forms: 1,2-diacylglycerols and 1,3-diacylglycerols. Examples are glycerol dicaprate, glycerol dilaurate, glycerol dipalmitate, glycerol dimyristate and glycerol distearate, and mixed forms, such as glycerol lauratepalmitate, glycerol lauratestearate and the like.

A monoglyceride is an ester derived from glycerol and one fatty acid. Two possible forms exist: 1-acylglycerols and 2-acylglycerols. Examples are glycerol monolaurate, monopalmitate, monomyristate and monostearate.

Suitable fatty acids are for example lauric acid, palmitic acid, myristic acid or stearic acid.

A suitable steroid is for example cholesterol.

A suitable wax is for example cetyl palmitate.

The natural polymers are preferably selected from the group consisting of polysaccharides, in particular starch, cellulose, pullulan or dextran; polyaminosaccharides, in particular chitosan; and polypeptides, in particular proteins, specifically albumin.

The synthetic polymers are preferably selected from the group consisting of poly(meth)acrylates, polystyrenes, polyethylene glycols, polyethyleneimines and polyesters of hydroxycarboxylic acids.

The term “poly(meth)acrylates” denotes either polyacrylates or polymethacrylates or mixtures thereof or copolymers of acrylates and methacrylates. Acrylates and methacrylates are the esters of acrylic and methacrylic acid, respectively.

In order to offer a reaction site at which the albumin (iii) can be bound covalently to the material (ii), either directly or via a linking group, poly(meth)acrylates to be used as material (ii) suitably carry a functional group to which the albumin (iii) or a linking group for the albumin can bind, or which can be converted into a functional group to which the albumin (iii) or a linking group therefor can bind. If the albumin is to be bound directly to the poly(meth)acrylate, the functional group on the poly(meth)acrylate has to be one which can react with the amino groups of the albumin under mild conditions in order to avoid denaturation of the albumin. One suitable functional group for this purpose is the carboxyl group which can react with amino groups of the albumin to carboxyamide groups. Amide formation under mild conditions can be carried out, for example, by using suitable activators.

Thus, suitable poly(meth)acrylates for this purpose are polymers which, in addition to (meth)acrylic esters, contain unsaturated carboxylic acids in copolymerized form. Suitable unsaturated carboxylic acids are acrylic acid, methacrylic acid, crotonic acid, maleic acid, fumaric acid and itaconic acid. Preference is given to acrylic acid and methacrylic acid.

Another suitable functional group for this purpose is the sulfonic acid group which can react with amino groups of the albumin to sulfonamide groups. Thus, suitable poly(meth)acrylates are polymers which, in addition to (meth)acrylic esters, contain unsaturated carboxylic acids in copolymerized form. Examples are esters of acrylic or methacrylic acid derived from alcohols which contain sulfonic acid groups.

If the albumin is not to be bound directly to the poly(meth)acrylate, but via a linking group, the functional group on the poly(meth)acrylate can be varied largely, since the functional group is generally first reacted with a linking group before the more sensitive albumin comes into play. The functional group can be bound to that part of the (meth)acrylate molecule which is derived from the alcohol, or to a carbon atom of the original C—C double bond. The functional group bound to that part of the (meth)acrylate molecule which is derived from the alcohol can for example be selected from the group consisting of cyano, azido, hydroxyl, amino, thiol, carbonyl, carboxyl, sulfonic acid, sulfonates, such as tosylate, triflate or nonaflate, a C—C double bond or a C—C triple bond, to name just a few. The functional group bound to a carbon atom of the original C—C double bond can for example be selected from the group consisting of cyano, carbonyl, carboxyl, a C—C double bond or a C—C triple bond.

Examples of such functionalized (meth)acrylates are hydroxyalkyl(meth)acrylates, such as 2-hydroxyethylacrylate, 2-hydroxyethylmethacrylate, 3-hydroxypropylacrylate, 3-hydroxypropylmethacrylate, 4-hydroxybutylacrylate, 4-hydroxybutylmethacrylate and the like; aminoalkyl(meth)acrylates, such as 2-aminoethylacrylate, 2-aminoethylmethacrylate, 3-aminopropylacrylate, 3-aminopropylmethacrylate, 4-aminobutylacrylate, 4-aminobutylmethacrylate and the like, maleic acid, fumaric acid, citraconic acid, alkylcyanoacrylates, such as butylcyanoacrylates and the like.

The polymers can be homopolymers of said functionalized (meth)acrylates or copolymers containing said functionalized (meth)acrylates and alkyl(meth)acrylates in copolymerized form.

Preference is given to poly(butylcyanoacrylates), especially to poly(butylcyanoacrylates) as described in WO 2017/084854, WO 2017/085212 or the references cited therein.

The poly(butylcyanoacrylates) may contain a further functionalization which is derived from the reaction of the acidic hydrogen atom bound to that carbon atom which carries the C(O)O-butyl and the CN group. This acidic H can be reacted with an alkyl halide in which the alkyl group carries a functional group, such as those listed above, or with an alkenyl halide. One example is the reaction with ethyl 2-(bromomethyl) acrylate, as described in WO 2017/084854.

As regards polystyrenes, analogous thoughts apply: In order to offer a reaction site at which the albumin (iii) can be bound covalently to the material (ii), either directly or via a linking group, polystyrenes to be used as material (ii) suitably carry a functional group to which the albumin (iii) or a linking group for the albumin can bind, or which can be converted into a functional group to which the albumin (iii) or a linking group therefor can bind. If the albumin is to be bound directly to the polystyrenes, the functional group on the polystyrenes has to be one which can react with the amino groups of the albumin under mild conditions in order to avoid denaturation of the albumin. One suitable functional group for this purpose is the carboxyl group which can react with amino groups of the albumin to carboxyamide groups. Amide formation under mild conditions can be carried out by using suitable activators.

Examples for suitable polystyrenes functionalized with carboxy groups are copolymers of styrene with acrylic acid or methacrylic acid.

If the albumin is not to be bound directly to the polystyrenes, but via a linking group, copolymers of styrene with one or more of the above monomers can be used, e.g. with hydroxyalkyl(meth)acrylates, such as 2-hydroxyethylacrylate, 2-hydroxyethylmethacrylate, 3-hydroxypropylacrylate, 3-hydroxypropylmethacrylate, 4-hydroxybutylacrylate, 4-hydroxybutylmethacrylate and the like; aminoalkyl(meth)acrylates, such as 2-aminoethylacrylate, 2-aminoethylmethacrylate, 3-aminopropylacrylate, 3-aminopropylmethacrylate, 4-aminobutylacrylate, 4-aminobutylmethacrylate and the like, or alkylcyanoacrylates, such as butylcyanoacrylates.

Examples of polyesters of hydroxycarboxylic acids are poly(lactic acid), poly(glycolic acid), poly(lactic glycolic acid), poly-3-hydroxybutyrate (PHB), poly-3-hydroxybutyrate-co-3-hydroxyvalerate (PHBV), poly-3-hydroxybutyrate-co-3-hydroxyhexanoate (PHBHHx) or poly-(3-hydoxybutyrate-co-3-hydroxy octanoate) (PHBHO).

Carbon nanotubes (CNTs) are allotropes of carbon with a cylindrical nanostructure and are members of the fullerene structural family. Their name is derived from their long, hollow structure with the walls formed by one-atom-thick sheets of carbon, i.e. by graphene. These sheets are rolled at specific and discrete (“chiral”) angles, and the combination of the rolling angle and radius decides the nanotube properties. Carbon nanotubes are generally categorized as single-walled carbon nanotubes (SWCNTs; often just SWNTs) and multi-walled carbon nanotubes (MWCNTs; often just MWNTs). For the purpose of the present invention, both types are useful.

In a particular embodiment, lipids are used as material (ii). Among the lipids, preference is given to triglycerides, diglycerides, monoglycerides, fatty acids, steroids, and waxes. More preference is given to triglycerides, in particular to trilaurin, tripalmitin, trimyristin and tristearin. Specifically, trilaurin is used as material (ii).

### Albumin

The albumin (iii) is preferably serum albumin. To reduce or avoid immune reactions, the albumin (iii) is preferably a serum albumin of that species to which the subject (i.e., the human or non-human animal) that is to be brought into contact (e.g., to be treated) with the nanoparticle of the invention belongs. For example, the serum albumin can be selected from the group consisting of human serum albumin, bovine serum albumin, monkey serum albumin, especially rhesus macaque serum albumin, marmoset serum albumin, macaque serum albumin, e.g. cynomolgous monkey albumin, baboon serum albumin or katta serum albumin, dog serum albumin, rat serum albumin and mouse serum albumin. In particular, the albumin is human serum albumin or bovine serum albumin. Specifically, the albumin is human serum albumin.

### Targeting Ligands

Targeting ligands are ligands, e.g. small molecules or more complex structures, such as synthetic polymers, polypeptides or proteins, which interact with cell-specific or tissue-specific surface structures and allow for the nanoparticles to interact, e.g. bind, (relatively) specifically with/to the respective cell. Such cell-specific surface structures are for example cell surface proteins or lipids of the plasma membrane; examples being receptors, ion channels and ganglioside M1. The term “cell surface protein” includes all proteins of which at least a part is accessible on the cell surface, e.g. transmembrane proteins with extracellular domains.

In a preferred embodiment, the targeting ligand is a ligand targeting extracellular domains of transmembrane proteins or targeting cell surface proteins. In particular, the targeting ligand is a ligand targeting receptors or ion channels. Specifically, the targeting ligand is a ligand targeting a receptor; i.e. a receptor-targeting ligand.

Receptor-targeting ligands are ligands which that are capable of being recognized (i.e. specifically bound) by a receptor protein located in a cell membrane, for example a receptor of an endothelial cell at the blood-brain barrier that facilitates uptake into the endothelial cell and/or transcytosis into the brain parenchyma. The binding of the receptor-targeting ligand to the receptor protein can facilitate the uptake of the nanoparticles of the invention by a cell carrying the receptor protein in its cell membrane. Thus, the nanoparticles can be delivered to a specific organ or tissue and their uptake by the cells of said organ or tissue can be increased. This makes the nanoparticles of the present invention particularly suitable for uses in therapy and prophylaxis of disorders and diseases, wherein the cargo substance has to be delivered to specific sites within the body, for example across the blood-brain barrier that is usually not permeable to most pharmaceuticals.

Targeting ligands are principally known and described in numerous publications, such as in Oller-Salvia B, Sánchez-Navarro M, Giralt E, Teixidó M. Blood-brain barrier shuttle peptides: an emerging paradigm for brain delivery. Chem. Soc. Rev. 2016 Aug. 22; 45(17):4690-707; Julia V. Georgieva, Dick Hoekstra, and Inge S. Zuhorn. Smuggling Drugs into the Brain: An Overview of Ligands Targeting Transcytosis for Drug Delivery across the Blood-Brain Barrier. Pharmaceutics. 2014 Dec.; 6(4): 557-583 or Gao H. Progress and perspectives on targeting nanoparticles for brain drug delivery. Acta Pharm Sin B. 2016 July; 6(4):268-86.

Examples for small molecules as targeting ligands are vitamins such as folic acid or the corresponding folate anion and thiamin.

Examples for targeting ligands of a larger structure are synthetic polymers, peptides, proteins, and deoxyribonucleic acids (DNAs, such as aptamers targeting cell- or tissue-specific surface structures).

The synthetic polymers to be used in the context of the present invention are expediently biocompatible, i.e. do not cause inacceptable toxicity or side effects when thus used. Examples are polyoxyalkylene-containing polymers, such as polyoxyethylene-polyoxypropylene copolymers or polysorbates.

Suitable polyoxyethylene-polyoxypropylene copolymers are for example the poloxamers, which are triblock copolymers composed of a central polyoxypropylene (poly(propylene oxide)) block flanked by two chains of polyoxyethylene (poly(ethylene oxide) blocks, for instance Poloxamer 188 (poloxamer with a polyoxypropylene molecular mass of ca. 1800 g/mol and ca. 80% by weight polyoxyethylene content) or Poloxamer 407 (poloxamer with a polyoxypropylene molecular mass of ca. 4,000 g/mol and ca. 70% by weight polyoxyethylene content).

Polysorbates are polyoxyethylene sorbitan monoesters and triesters with monounsaturated or, in particular, saturated fatty acids. Examples of particular fatty acids include, but are not limited to, C₁₁-C₁₈-fatty acids such as lauric acid, palmitic acid, stearic acid and, in particular, oleic acid. The polyoxyethylene sorbitan fatty acid esters may comprise up to 90 oxyethylene units, for example 15-25, 18-22 or, preferably, 20 oxyethylene units. They are preferably selected from polyoxyethylene sorbitan fatty acid esters having an HLB value in the range of about 13-18, in particular about 16-17. Expediently, polysorbates are selected from officially approved food and/or drug additives such as, for example, polysorbate 20 (E432; polyoxyethylene (20) sorbitan monolaurate), polysorbate 40 (E434; polyoxyethylene (20) sorbitan monopalmitate), polysorbate 60 (E435; polyoxyethylene (20) sorbitan monostearate), polysorbate 65 (E436) and polysorbate 80 (E433; polyoxyethylene (20) sorbitan monooleate). “Polyoxyethylene 20” means an average of 20 oxyethylene —(CH₂CH₂O)— repeating units per molecule. Specifically, the polysorbate is polysorbate 80.

Examples for peptides that can be used as targeting ligands in the context of the present invention are:
Angiopep-2 (TFFYGGSRGKRNNFKTEEY; SEQ ID NO:3)
ApoB (3371-3409) (SSVIDALQYKLEGTTRLTRKRGLKLATALSLSNKFVEGS; SEQ ID NO:4)
ApoE (159-167)₂ ((LRKLRKRLL)₂; SEQ ID NO:5)
Peptide-22 (Ac-C(&)MPRLRGC(&)-NH₂; cysteines marked as “C(&)” are linked via a disulfide bond; C-terminus amidated; SEQ ID NO:6)
transferrin receptor binding-peptides, e.g. THR (THRPPMWSPVWP-NH₂; C-terminus amidated; SEQ ID NO:7) and retro-enantio THR (pwvpswmpprht-NH₂, amino acids in lowercase letter are D-amino acids) (Lee J H, Engler J A, Collawn J F, Moore B A. Receptor mediated uptake of peptides that bind the human transferrin receptor. Eur J Biochem. 2001 April; 268(7):2004-12)
CRT (C(&)RTIGPSVC(&); cysteines marked as “C(&)” are linked via a disulfide bond; SEQ ID NO:8)
Leptin30 (YQQILTSMPSRNVIQISNDLENLRDLLHVL; SEQ ID NO:9)
Acetylcholine receptor-binding domain of RVG (RVG29; YTIWMPENPRPGTPCDIFTNSRGKRASNG; SEQ ID NO:2)
^{D}CDX (greirtgraerwsekf; amino acids in lowercase letter are D-amino acids)
Apamin (C(&1)NC(&2)KAPETALC(&1)ARRC(&2)QQH-NH₂; cysteines marked as “C(&1)” are linked via a disulfide bond; cysteines marked as “C(&2)” are linked via a disulfide bond; C-terminus amidated; SEQ ID NO:10)
MiniAp-4 ([Dap](&)KAPETALD(&); N- and C-terminus of the peptide are linked via diaminopropyl (Dap); SEQ ID NO:11)
reduced glutathione (GSH; gamma-L-glutamyl-L-cysteinylglycine)
G23 (HLNILSTLWKYRC; SEQ ID NO12)
G7 (GFtGFLS(O-beta-Glc)-NH₂; C-terminus amidated; amino acid “t” is D-threonine; SEQ ID NO:13)
TGN (TGNYKALHPHNG; SEQ ID NO:14)
TAT (47-57) (YGRKKRRQRRR-NH₂; C-terminus amidated; SEQ ID NO:15)
SynB1 (RGGRLSYSRRRFSTSTGR; SEQ ID NO:16)
diketopiperazines (&(N-MePhe)-(N-MePhe)Diketopiperazines) (Teixidó M, Zurita E, Malakoutikhah M, Tarragó T, Giralt E. Diketopiperazines as a tool for the study of transport across the blood-brain barrier (BBB) and their potential use as BBB-shuttles. J Am Chem Soc. 2007 Sep. 26; 129(38):11802-13; and Teixidó M, Zurita E, Mendieta L, Oller-Salvia B, Prades R, Tarragó T, Giralt E. Dual system for the central nervous system targeting and blood-brain barrier transport of a selective prolyl oligopeptidase inhibitor. Biopolymers. 2013 November; 100(6):662-74)
PhPro ((Phenylproline)₄-NH₂; C-terminus amidated; SEQ ID NO:17)
EPRNEEK (EPRNEEK; SEQ ID NO:18)
chlorotoxin (originating from Leiurus quinquestriatus;
   MC(&1)MPC(&2)FTTDHQMARKC(&3)DDC(&1) C(&4)GGKGRGKC(&2)YGPQC(&3)LC(&4)R-NH₂; cysteines marked as “C(&1)” are linked via a disulfide bond; cysteines marked as “C(&2)” are linked via a disulfide bond; cysteines marked as “C(&3)” are linked via a disulfide bond; cysteines marked as “C(&4)” are linked via a disulfide bond; C-terminus amidated; SEQ ID NO:19)
   insulin (e.g., amino acid sequence set forth in GenBank accession no. V00565.1); and
peptides derived from tetanus toxin.

Examples for proteins are
transferrin (e.g., as encoded by the polynucleotide sequence set forth in M12530.1 (mRNA) or AY308797.1 (genomic DNA))
apolipoprotein E3 (ApoE3) (e.g., as encoded by the polynucleotide sequence set forth in GenBank accession no. FJ525876.1 (DNA))
apolipoprotein A1 (ApoA1) (e.g., as encoded by the polynucleotide sequence set forth in GenBank accession no. J00098.1 (DNA))
apolipoprotein B100 (ApoB100) (e.g., as encoded by the polynucleotide sequence set forth in GenBank accession no. AH003569.2 (DNA))
antigen-binding molecules; in particular antibodies, antigen-binding fragments thereof, molecules comprising at least one antigen-binding region of an antibody, or antibody mimetics targeting cell- or tissue-specific surface structures
tetanus toxin (e.g., amino acid sequence set forth in GenBank accession no. X04436.1)
CRM197 (non-toxic analog of the diphteria toxin; e.g., amino acid sequence set forth in GenBank accession no. X00703.1)
rabies virus glycoprotein (transmembrane glycoprotein G, e.g., amino acid sequence set forth in Genbank M13215.1).

The above-mentioned peptides and proteins having sequences found in naturally occurring sources, such as e.g. transferrin, apolipoprotein, insulin, etc., may exhibit inter- and intraspecies variants. Unless further specified, the designations of said proteins and peptides are meant to refer to all of such variants. Preferably, said proteins and peptides are from the same species as the subject to be treated with the nanoparticles of the invention carrying such protein or peptide as targeting ligand.

The term “antigen-binding molecules”, as used herein, refers to antibodies, antigen-binding fragments thereof, molecules comprising at least one antigen-binding region of an antibody as well as to antibody mimetics.

The antigen-binding molecules can be polyclonal or monoclonal antibodies, with monoclonal antibodies being preferred. The antibodies may be naturally occurring antibodies or genetically engineered variants thereof. The antibodies may be selected from the group consisting of avian (e.g. chicken) antibodies and mammalian antibodies (e.g. human, murine, rat or cynomolgus antibodies), with human antibodies being preferred. The antibodies can be chimeric such as, for example, chimeric antibodies derived from murine antibodies by exchange of part or all of the non-antigen-binding regions by the corresponding human antibody regions. Where the antibody is a mammalian antibody, it may belong to one of several major classes including IgA, IgD, IgE, IgG, IgM and heavy chain antibodies (as found in camelids). IgGs (gammaglobulins) are the preferred class if mammalian antibodies because they are the most common antibodies in mammals, are specifically recognized by Fc gamma receptors and can generally be easily prepared in vitro. Where the antibody is an IgG, it may belong to one of several isotypes including IgG1, IgG2, IgG3 and IgG4. The antibodies can be prepared, for example, via immunization of animals, via hybridoma technology or recombinantly.

The antigen-binding molecules can be antigen-binding fragments of antibodies such as, for example, Fab, F(ab)₂ and Fv fragments.

The antigen-binding molecules can be molecules having at least one antigen-binding region of an antibody which can be selected from the group consisting of, but are not limited to, dimers and multimers of antibodies; bispecific antibodies; single chain Fv fragments (scFv) and disulfide-coupled Fv fragments (dsFv).

The antigen-binding molecules can also be antibody mimics. The term “antibody mimics”, as used herein, refers to artificial polypeptides or proteins which are capable of binding specifically to an antigen but are not structurally related to antibodies. For example such polypeptides and proteins may be based on scaffolds such as the Z domain of protein A (i.e. affibodies), gamma-B crystalline (i.e. affilins), ubiquitin (i.e. affitins), lipcalins (i.e. anticalins), domains of membrane receptors (i.e. avimers), ankyrin repeat motif (i.e. DARPins), the 10^{th} type III domain of fibronectin (i.e. monobodies). The term “antibody mimics” also includes dimers and multimers of such polypeptides or proteins.

The above-listed and other suitable targeting peptides or proteins can comprise or basically consist of natural peptide or protein ligands for cell membrane-located receptor proteins and receptor-recognized portions of said peptide/protein ligands. Examples of receptor-recognized portions of natural peptide or protein ligands include, but are not limited to, the peptides of SEQ ID NOs:1-2.

LDL receptor-binding domain of ApoE4
(SEQ ID NO: 1)
Tyr-Leu-Arg-Val-Arg-Leu-Ala-Ser-His-Leu-Arg-Lys-
Leu-Arg-Lys-Arg-Leu-Leu-Arg-Asp-Ala-Asp-Asp-Leu-
Tyr
Acetylcholine receptor-binding domain of RVG
(SEQ ID NO: 2)
Tyr-Thr-Ile-Trp-Met-Pro-Glu-Asn-Pro-Arg-Pro-Gly-
Thr-Pro-Cys-Asp-Ile-Phe-Thr-Asn-Ser-Arg-Gly-Lys-
Arg-Ala-Ser-Asn-Gly

Alternatively, suitable targeting peptides/proteins can comprise or basically consist of synthetic peptide or protein ligands for cell membrane-located receptor proteins. Examples of synthetic ligands for cell membrane-located receptor proteins include, but are not limited to, the peptide of SEQ ID NO:3.

(SEQ ID NO: 3)
Thr-Phe-Phe-Tyr-Gly-Gly-Ser-Arg-Gly-Lys-Arg-Asn-
Asn-Phe-Lys-Thr-Glu-Glu-Tyr

In a preferred embodiment, the targeting ligand is selected from the group consisting of vitamins, in particular the above-listed vitamins, synthetic polymers, specifically polyoxyalkylene-containing polymers, in particular the above-listed polyoxyalkylene-containing polymers, peptides, in particular the above-listed peptides, and proteins, in particular the above-listed proteins.

Specifically, the targeting ligand is transferrin.

### Linker

In a preferred embodiment, the linker via which the targeting ligand is covalently bound to the albumin (iii) contains one or more polyalkyleneoxide chains, in particular one or more polyethyleneglycol chains (containing —CH₂CH₂—O— as repeating units), where the polyalkyleneoxide chains contain an overall amount of alkylene oxide repeating units of from 10 to 500, in particular of from 20 to 200.

“Overall amount” of alkylene oxide repeating units adumbrates to the fact that the polyalkyleneoxide chain of the linker can be interrupted by one or more groups different from alkyleneoxide-derived moieties. These groups generally stem from the synthetic method via which albumin, linker and targeting ligand are connected. For example it might be expedient to link first the albumin to a part of the polyalkyleneoxide chain and the targeting ligand to the other part and then link the two chain parts via another molecule.

### Further Components

The nanoparticles can comprise further components.

The nanoparticles of the invention can comprise one or more than one nanoparticle-stabilizing agent selected from the group consisting of bile acids (e.g. cholic acid, taurocholic acid, glycocholic acid, deoxycholic acid, lithocholic acid, chenodeoxycholic acid, dehydrocholic acid, ursodeoxycholic acid, hyodeoxycholic acid and hyocholic acid), salts (e.g. sodium, potassium or calcium salts) of bile acids, and mixtures thereof.

The nanoparticle of the invention may moreover contain a detectable moiety. Suitable detectable moieties include, but are not limited to, fluorescent moieties and moieties which can be detected by an enzymatic reaction or by specific binding of a detectable molecule (e.g. a fluorescence-labelled antibody). Fluorescent moieties are for example fluorescein, rhodamine B or 5-(and-6)-carboxyrhodamine (5(6)-CR 110). The detectable moiety can for example be bound to the cargo substance, especially if this is a biopharmaceutical, or can be bound to the material (ii) or can be bound to the albumin or to the targeting ligand.

### Method for Producing the Nanoparticles

In another aspect, the present invention relates to a method for producing the nanoparticles of the invention, which method comprises
(a) providing a nanoparticle in which a cargo substance (i) is surrounded by or embedded in the material (ii);
(b) if necessary, modifying the material (ii) of the nanoparticle of step (a) in such a way that it can covalently bind the albumin (iii) either directly or via a linking group A;
(c) covalently attaching to the optionally modified nanoparticle
   (c.1) the albumin; or
   (c.2) the linking group A via which the albumin is to be attached to the optionally modified nanoparticle; or
   (c.3) the linking group A to which the albumin is already attached; or
   (c.4) the albumin which carries the covalently bound linker via which the targeting ligand is to be bound, or a part of the linker; or
   (c.5) the albumin which carries the covalently bound linker to which the targeting ligand is attached; or
   (c.6) the linking group A to which the albumin is already attached, where the albumin carries moreover the covalently bound linker via which the targeting ligand is to be bound, or a part of the linker; or
   (c.7) the linking group A to which the albumin is already attached, where the albumin carries moreover the covalently bound linker to which the targeting ligand is attached;
(d.1) in case that step (c) is step (c.2), attaching to the linking group A of the product obtained in step (c.2)
   (d.1.1) the albumin; or
   (d.1.2) the albumin which carries the covalently bound linker via which the targeting ligand is to be bound, or a part of the linker; or
   (d.1.3) the albumin which carries the covalently bound linker to which the targeting ligand is attached;
(d.2) in case that step (c) is step (c.1) or (c.3) and in case that step (d.1) is step (d.1.1), attaching to the albumin of the product obtained in step (c.1), (c.3) or (d.1.1)
   (d.2.1) the linker or a part thereof; if necessary by reacting the albumin first with a linking group B and then with the linker or a part thereof; or
   (d.2.2) the linker which already carries the targeting ligand; if necessary by reacting the albumin first with a linking group B and then with the linker already carrying the targeting ligand;
(e.1) in case that step (c) is step (c.4) or (c.6) and in case that step (d.1) is step (d.1.2) and in case that step (d.2) is step (d.2.1), for the case that only a part of the linker is contained in the product obtained in step (c.4), (c.6) (d.1.2) or (d.2.1), either
   (e.1.1) converting the part of the linker into the complete linker; or
   (e.1.2) reacting the part of the linker with the rest of the linker to which the targeting ligand is already attached; and
(e.2) in case that step (c) is step (c.4) or (c.6) and in case that step (d.1) is step (d.1.2) and in case that step (d.2) is step (d.2.1), for the case that the complete linker is contained in the product obtained in step (c.4), (c.6) (d.1.2) or (d.2.1), and in case that step (e.1) is step (e.1.1), attaching the targeting ligand to the linker.

Methods for carrying out step (a) are principally known in the art or can be adapted from known methods. The optimum way depends of course on the cargo substance (i) and the material (ii), but can be adapted from known methods by those skilled in the art.

Nanoparticles where the material (ii) is a lipid and the cargo substance (i) is stable in aqueous medium can for example be prepared as detailed below.

Nanoparticles where the material (ii) is a poly(meth)acrylate can for example be prepared in analogy to the methods described in WO 2017/084854, WO 2017/085212 or the references cited therein.

Nanoparticles where the material (ii) is a synthetic polymer and the cargo substance (i) is not susceptible to degradation under harsher reaction conditions can moreover be prepared by polymerizing the monomers from which the polymeric material, i.e. the polymeric shell (in case of nanocapsules) or polymer matrix (in case of matrix particles) is to be formed, or polymerizing or curing a pre-polymer or pre-condensate from which the polymeric material, i.e. the polymeric shell (in case of nanocapsules) or polymer matrix (in case of matrix particles) is to be formed, in the presence of the cargo substance (i).

Polymerization can for example be carried out as an interfacial polymerization process of a suitable polymer wall forming material. Interfacial polymerization is usually performed in an aqueous oil-in-water emulsion or suspension of the core material containing dissolved therein at least one part of the polymer wall forming material. During the polymerization, the polymer segregates from the core material to the boundary surface between the core material and water thereby forming the wall of the nanocapsule. Thereby an aqueous suspension of the nanocapsule material is obtained.

Polymerization of (meth)acrylates or styrenes to prepare nanocapsules with a poly(meth)acrylate or polystyrene shell can for example be prepared starting from an oil-in-water emulsion of the monomers, the cargo substance (i) and suitably also a protective colloid. Polymerization of the monomers is then triggered by addition of a free radical starter and optionally also by heating and if appropriate controlled through a further temperature increase. The resulting polymers form the capsule wall which surrounds the core substance. This general principle is described for example in WO 2008/071649 or DE-A-10 139 171.

Curing of a pre-polymer or pre-condensate can be effected or initiated in a manner well-known for the respective pre-polymer or pre-condensate, e.g. by heating an aqueous dispersion thereof to a certain reaction temperature, adding curing agents or changing the pH.

The above polymerization and curing methods are however generally not applicable when the cargo substance (i) is a biopharmaceutical, since these are generally susceptible to degradation under most polymerization or curing conditions.

Step (b), i.e., modifying the material (ii) of the nanoparticle of step (a) in such a way that it can covalently bind the albumin (iii) either directly or via a linking group A, becomes necessary if the material (ii) of the nanoparticle obtained in step (a) does not contain any group to which the albumin or a linking group A can bind.

Albumin generally reacts via one or more of its amino groups. A typical reaction of amino groups to form new covalent bonds and which can occur under mild conditions is the formation of carboxamide groups or sulfonamide groups. Thus, step (b) is not necessary if material (ii) of the nanoparticle of step (a) contains carboxyl (C(O)OH) or sulfonic acid groups or contains an activated carboxyl group.

In the former case (material (ii) of the nanoparticle of step (a) contains carboxyl (C(O)OH) or sulfonic acid groups) the amide formation has to carried out in the presence of an activator (coupling agent). Suitable coupling reagents (activators) are well known and are for instance selected from the group consisting of carbodiimides, such as DCC (dicyclohexylcarbodiimide), DCI (diisopropylcarbodiimide) and EDCI (1-ethyl-3-(3-dimethylaminopropyl)carbodiimide), benzotriazol derivatives, such as HATU (O-(7-azabenzotriazol-1-yl)-N,N,N′,N′-tetramethyluronium hexafluorophosphate), HBTU ((O-benzotriazol-1-yl)-N,N,N′,N′-tetramethyluronium hexafluorophosphate) and HCTU (1H-benzotriazolium-1-[bis(dimethylamino)methylene]-5-chloro tetrafluoroborate) and phosphonium-derived activators, such as BOP ((benzotriazol-1-yloxy)-tris(dimethylamino)phosphonium hexafluorophosphate), Py-BOP ((benzotriazol-1-yloxy)-tripyrrolidinphosphonium hexafluorophosphate) and Py-BrOP (bromotripyrrolidinphosphonium hexafluorophosphate).

In the latter case (material (ii) of the nanoparticle of step (a) contains an activated carboxyl group) the use of activators is not necessary. Activated carboxyl groups are for example activated esters formally obtained from the reaction of a carboxyl group with an active ester-forming alcohol, such as p-nitrophenol, N-hydroxybenzotriazole (HOBt), N-hydroxysuccinimide, N-hydroxysuccinimide carrying a sulfonic acid group or OPfp (pentafluorophenol).

Groups within the material (ii) to which a linking group A can bind can vary widely. They can for example be selected from the group consisting of cyano, azido, hydroxyl, amino, thiol, carbonyl, carboxyl, sulfonic acid, sulfonates, such as tosylate, triflate or nonaflate, a C—C double bond or a C—C triple bond, to name just a few. The linking group A molecule has of course to have a group which can react with such a functional group to a covalent bond. If the linking group A is not yet bound to the albumin, the reactions between the functional group within the material (ii) and functional group within the linking group molecule A can vary in extenso. Just by way of example,
a cyano group within the material (ii) can be reduced to a primary amino group and then reacted with a carboxyl, sulfonic acid or sulfonate group of the linking group A molecule; or can be reacted with a sulfonate group to a secondary amino group;
a cyano group within the material (ii) can be hydrolyzed to a carboxyl group and then be reacted with a hydroxy, thio or primary or secondary amino group of the linking group A molecule to an ester, carboxamide or thiocarboxamide group;
an azido group within the material (ii) can be reacted in a click reaction with a strained C—C triple bond of the linking group A molecule to a triazole moiety;
an azido group within the material (ii) can be reacted in a click reaction with a terminal C—C triple bond of the linking group A molecule in the presence of a Cu catalyst to a triazole moiety;
a hydroxyl, primary or secondary amino or thiol group within the material (ii) can be reacted with a carboxyl or sulfonic acid group of the linking group A molecule to a carboxylic ester, carboxamide, thiocarboxamide, sulfonate, sulfonamide or thiosulfonate group;
a carbonyl group within the material (ii) can be reacted with a primary amino group of the linking group A molecule to an imine and then be reduced to a secondary amino group;
a carbonyl group within the material (ii) can be reacted with a primary amino group of the linking group A molecule to an imine and then be reduced to a secondary amino group;
a sulfonate group within the material (ii) can be reacted with a hydroxyl group, a primary or secondary amino group or a thiol group of the linking group A molecule to an ether, secondary or tertiary amino group or a thioether group;
a C—C double within the material (ii) bond can be reacted in an addition reaction, e.g. to a thiol-ene-click reaction by reaction with a thiol group of the linking group A molecule, especially if the double bond is part of a Michael system, i.e. bound to a carbonyl group; or with a hydroxy group thereof;
a C—C double within the material (ii) bond can be reacted in a [2+3]-cycloaddition reaction, e.g. with an azide group of the linking group A molecule;
a C—C double within the material (ii) bond can be reacted in a [2+4]-cycloaddition reaction, e.g. with a butadiene-derived moiety (i.e. two conjugated C—C double bonds) in the linking group A molecule, to a cyclohexene moiety;
a C—C double within the material (ii) bond can be reacted in a click reaction with a tetrazine in the linking group A molecule to a dihydropyridine;
a terminal or strained C—C triple bond within the material (ii) can be reacted in a click reaction with an azide group of the linking group A molecule to a triazole moiety; if the triple bond is terminal, the reaction has to be carried out in the presence of a catalyst, generally a Cu catalyst.

In material (ii) of the nanoparticle obtained in step (a) contains no functional group to which a linking group A can bind, it has to be modified accordingly, e.g. by oxidation, hydrolysis, amination or other processes known in the art as suitable for the respective material (ii). Generally, however, material (ii) is chosen or formed from the beginning in such a way that it contains suitable functional groups.

Suitable conditions for steps (c.1) (covalently attaching to the optionally modified nanoparticle the albumin), (c.4) (covalently attaching to the optionally modified nanoparticle the albumin which carries the covalently bound linker via which the targeting ligand is to be bound, or a part of the linker) and (c.5) (covalently attaching to the optionally modified nanoparticle the albumin which carries the covalently bound linker to which the targeting ligand is attached) have already been depicted above: Albumin generally reacts via one or more of its amino groups. A typical reaction of amino groups to form new covalent bonds and which can occur under mild conditions is the formation of carboxamide groups or sulfonamide groups. Thus, an expedient way to carry out step (c.1), (c.4) or (c.5) is to react albumin with carboxyl (C(O)OH) or sulfonic acid groups or activated carboxyl groups of material (ii) in the optionally modified nanoparticle to yield carboxamide or sulfonamide groups.

As said, in the case of carboxyl (C(O)OH) or sulfonic acid groups, the amide formation has to be carried out in the presence of an activator (coupling agent). Suitable coupling reagents (activators) are listed above.

As said, activated carboxyl groups are for example activated esters formally obtained from the reaction of a carboxyl group with an active ester-forming alcohol, such as p-nitrophenol, N-hydroxybenzotriazole (HOBt), N-hydroxysuccinimide or OPfp (pentafluorophenol). The reaction of the albumin with such groups generally occurs spontaneously upon contact.

Step (c.2) (covalently attaching to the optionally modified nanoparticle the linking group A via which the albumin is to be attached to the optionally modified nanoparticle) can be carried out in various modes; the suitable reactions depending from the functional groups present in the material (ii) of the optionally modified nanoparticle obtained in step (a) or (b) and the linking group A molecule. As already listed above, following reactions are for example possible:
a cyano group within the material (ii) can be reduced to a primary amino group and then reacted with a carboxyl, sulfonic acid or sulfonate group of the linking group A molecule; or can be reacted with a sulfonate group to a secondary amino group;
a cyano group within the material (ii) can be hydrolyzed to a carboxyl group and then be reacted with a hydroxy, thio or primary or secondary amino group of the linking group A molecule to an ester, carboxamide or thiocarboxamide group;
an azido group within the material (ii) can be reacted in a click reaction with a strained C—C triple bond of the linking group A molecule to a triazole moiety;
an azido group within the material (ii) can be reacted in a click reaction with a terminal C—C triple bond of the linking group A molecule in the presence of a Cu catalyst to a triazole moiety;
a hydroxyl, primary or secondary amino or thiol group within the material (ii) can be reacted with a carboxyl or sulfonic acid group of the linking group A molecule to a carboxylic ester, carboxamide, thiocarboxamide, sulfonate, sulfonamide or thiosulfonate group;
a thiol group within the material (ii) can be reacted with a C—C double bond of the linking group A molecule in a thiol-ene-click reaction to a thioether group, especially if the double bond is part of a Michael system, i.e. bound to a carbonyl group;
a carbonyl group within the material (ii) can be reacted with a primary amino group of the linking group A molecule to an imine and then be reduced to a secondary amino group;
a sulfonate (leaving) group (such as triflate, nonaflate, tosylate) within the material (ii) can be reacted with a hydroxyl group, a primary or secondary amino group or a thiol group of the linking group A molecule to an ether, secondary or tertiary amino group or a thioether group;
a C—C double bond within the material (ii) bond can be reacted in an addition reaction, e.g. in a thiol-ene-click reaction by reaction with a thiol group of the linking group A molecule, especially if the double bond is part of a Michael system, i.e. bound to a carbonyl group; or with a hydroxy group thereof;
a C—C double bond within the material (ii) bond can be reacted in a [2+3]-cycloaddition reaction, e.g. with an azide group of the linking group A molecule;
a C—C or N-N double bond within the material (ii) bond can be reacted in a [2+4]-cycloaddition reaction ((hetero-)Diels-Alder reaction), e.g. with a butadiene-derived moiety (i.e. two conjugated C—C double bonds) in the linking group A molecule, to a cyclohexene moiety;
a C—C double bond within the material (ii) bond can be reacted in a click reaction with a tetrazine in the linking group A molecule to a dihydropyridine;
a terminal or strained C—C triple bond within the material (ii) can be reacted in a click reaction with an azide group of the linking group A molecule to a triazole moiety; if the triple bond is terminal, the reaction has to be carried out in the presence of a catalyst, generally a Cu catalyst.

Other reactions are also possible.

For carrying out step (c.3) (covalently attaching to the optionally modified nanoparticle the linking group A to which the albumin is already attached), (c.6) (covalently attaching to the optionally modified nanoparticle the linking group A to which the albumin is already attached, where the albumin carries moreover the covalently bound linker via which the targeting ligand is to be bound, or a part of the linker) or (c.7) (covalently attaching to the optionally modified nanoparticle the linking group A to which the albumin is already attached, where the albumin carries moreover the covalently bound linker to which the targeting ligand is attached) only such reactions are expedient which can be carried out in aqueous medium and which proceed under mild conditions (reaction temperature of at most 50° C., no strong acidic or basic media, no metal catalysis), so that the albumin is essentially not denaturated. Suitable reactions are for example:
an azido group within the material (ii) can be reacted in a click reaction with a strained C—C triple bond of the linking group A molecule to a triazole moiety;
a primary or secondary amino group within the material (ii) can be reacted with a carboxyl or sulfonic acid group of the linking group A molecule to a carboxamide or sulfonamide group in the presence of an activator;
a carboxyl or sulfonic acid group within the material (ii) can be reacted with a primary or secondary amino group of the linking group A molecule to a carboxamide or sulfonamide group in the presence of an activator;
a thiol group within the material (ii) can be reacted with a C—C double bond of the linking group A molecule in a thiol-ene-click reaction to a thioether group, especially if the double bond is part of a Michael system, i.e. bound to a carbonyl group;
a C—C double bond within the material (ii) bond can be reacted in an addition reaction, e.g. in a thiol-ene-click reaction by reaction with a thiol group of the linking group A molecule, especially if the double bond is part of a Michael system, i.e. bound to a carbonyl group;
a C—C double bond within the material (ii) bond can be reacted in a click reaction with a tetrazine in the linking group A molecule to a dihydropyridine;
a strained C—C triple bond within the material (ii) can be reacted in a click reaction with an azide group of the linking group A molecule to a triazole moiety;
an activated C—C or N—N double bond within the material (ii) bond can be reacted in a [2+4]-cycloaddition reaction ((hetero-)Diels-Alder reaction), e.g. with a butadiene-derived moiety (i.e. two conjugated C—C double bonds) in the linking group A molecule, to a cyclohexene moiety. C—C activated double bonds are e.g. those carrying in both α-positions a carbonyl group, such as in a maleic ester, acid, anhydride, amide or imide group. Activated N—N double bonds are e.g. those carrying in both α-positions a carbonyl group, such as in 1,3,4-triazolin-2,5-diones.

The reaction conditions for steps (d.1) and (d.2) are analogous to those for steps (c.1), (c.4) and (c.5). In case of step (d.1), it is the linking group A which has to carry a carboxyl group or a sulfonic acid group or an active ester group, and in case of step (d.2), it is the linker or a part thereof or the linking group B which has to carry a carboxyl group or a sulfonic acid group or an active ester group.

The reaction conditions for step (e.1.1) (converting the part of the linker into the complete linker) and (e.1.2) (reacting the part of the linker with the rest of the linker to which the targeting ligand is already attached) depend on the functional groups contained in the linker parts. They can for example be any of the reactions mentioned for step (c.3)

The reaction conditions for step (e.2) attaching the targeting ligand to the linker depend on the nature of the targeting compound. If this is for example a peptide or protein, suitable reaction conditions are those described for step (c.1), (c.4) or (c.5). Peptides and proteins generally react via their amino groups. Thus, the linker suitably carries a carboxyl group or a sulfonic acid group or an active ester group which reacts with the amino groups of the peptides or proteins to a carboxamide or sulfonamide group. If the targetting ligand is a polyoxyalkylene-containing polymer, these generally contain a terminal hydroxy group which can react for example with a sulfonate group in the linker to give an ether group or with a carboxyl group or an active ester group to give an ester group.

The following illustrates the method of the invention in more detail for the case that the material (ii) is a lipid and the cargo substance is stable in water:

For providing in step (a) a nanoparticle in which the cargo substance (i) which is stable in water and which is surrounded by or embedded in a lipid material (ii) and for modifying the material (ii) of the nanoparticle in such a way that it can covalently bind the albumin (iii), following steps can particularly be taken:
(a.1) a lipid, a functionalized lipid and one or more surfactants are dissolved in an organic solvent;
(a.2) the solution obtained in step (a.1) is mixed with a solution of the cargo substance in water to give a water-in-oil emulsion; and
(a.3) the water-in-oil emulsion obtained in step (a.2) is transferred to an aqueous phase to give a water-in-oil-in-water double emulsion.

The lipid corresponds to those defined above.

A functionalized lipid is a lipid which carries a functional group suitable for the reaction with a substance suitable to link the lipid and the albumin. A suitable functionalized lipid is for example a triglyceride in which one of the fatty acid residues is replaced by a group carrying a functional group. For example, the fatty acid residue can be replaced by a carboxylic acid residue carrying a further functional group or by a phosphate residue carrying a further functional group or by a sulfate residue carrying a further functional group. Suitable further functional groups depend on the intended reaction with the substance suitable to link the lipid and the albumin. Examples for couples of functional groups have been given above in context with step (ii). Such couples are for example
hydroxyl, primary or secondary amino (or precursor thereof, such a cyano group) or thiol group on the functionalized lipid/carboxyl, sulfonic acid or sulfonate group (the latter as leaving group; e.g. triflate, nonaflate, tosylate) on the substance suitable to link the lipid and the albumin; or vice versa carboxyl, sulfonic acid or sulfonate group (the latter as leaving group; e.g. triflate, nonaflate, tosylate) on the on the functionalized lipid/hydroxyl, primary or secondary amino (or precursor thereof, such a cyano group) or thiol group on the substance suitable to link the lipid and the albumin
azido group on the functionalized lipid/strained or terminal C—C triple bond on the substance suitable to link the lipid and the albumin; or vice versa strained or terminal C—C triple bond on the functionalized lipid/azido group on the substance suitable to link the lipid and the albumin
thiol group on the functionalized lipid/C—C double bond, especially C—C double bond bound to a carbonyl group, on the substance suitable to link the lipid and the albumin; or vice versa C—C double bond, especially C—C double bond bound to a carbonyl group, on the functionalized lipid/thiol group on the substance suitable to link the lipid and the albumin
carbonyl group on the functionalized lipid/primary amino group on the substance suitable to link the lipid and the albumin; or vice versa primary amino group on the functionalized lipid/carbonyl group on the substance suitable to link the lipid and the albumin
C—C double bond on the functionalized lipid/tetrazine on the substance suitable to link the lipid and the albumin; or vice versa tetrazine on the functionalized lipid/CC double bond on the substance suitable to link the lipid and the albumin
C—C double bond on the functionalized lipid/butadiene-derived moiety (i.e. two conjugated C—C double bonds) on the substance suitable to link the lipid and the albumin; or vice versa butadiene-derived moiety on the functionalized lipid/C—C double bond on the substance suitable to link the lipid and the albumin
N—N double bond on the functionalized lipid/butadiene-derived moiety on the substance suitable to link the lipid and the albumin; or vice versa/butadiene-derived moiety on the functionalized lipid/N—N double bond on the substance suitable to link the lipid and the albumin etc.

One example for such a functionalized lipid is a triglyceride in which one of the fatty acid groups is derived from a dicarboxylic acid, such as adipic acid. Another example is a triglyceride in which one of the fatty acid groups is derived from a hydroxycarboxylic acid, such as 4-hydroxybutyric acid. Another example is a triglyceride in which one of the fatty acid groups is derived from an aminocarboxylic acid, such as 4-aminobutyric acid. Another example is a triglyceride in which one of the fatty acid groups is derived from an unsaturated carboxylic acid with a double or triple bond. Another example is a phosphatidyl choline in which the amino group of the ethanol amine moiety is substituted by a moiety carrying a functional group. Examples for such a moiety carrying a functional group are groups of formula —C(═O)-A-X, where A is a bridging group, such as C₂-C₁₂-alkylene, preferably —(CH₂)ₙ—, where n is from 2 to 12, or —(CH₂CH₂—O)ₘ—, where m is from 1 to 6, and X is a functional group which can react with a functional group of the substance suitable to link the lipid and the albumin, e.g. an azido group (—N₃), —OH, —NH₂, —SH, —CH═CH₂, —C≡CH, —C(O)OH, —S(O)₂OH, —OS(O)₂CF₃ (triflate group) —OS(O)₂-(4-methylphenyl) (tosylate group), —C(O)H, —N(—C(O)—CH═CH—C(O)—) (N-bound maleimide group), —N(C(O)—N═N—C(O)—) (N-bound 1,3,4-triazoline-2,5-dione) and the like. One specific example for such a moiety carrying a functional group is the azidocaproyl group (—C(O)—CH₂)₆—N₃).

Specifically, the functionalized lipid is a phosphatidyl choline in which the amino group of the ethanol amine moiety is substituted by a 6-azidocaproyl group, in which the fatty acid residues in the glyceride moiety are C₁₂-C₂₀-fatty acid residues, such as lauroyl, myristoyl, palmitoyl, stearinoyl or arachinoyl. The functionalized lipid is thus specifically a compound CH₂(OR¹)—CH(OR²)—CH₂(OR³), where two of R¹, R² and R³ are a group —C(O)R⁴, where each R⁴ is independently C₁₁-C₁₉ alkyl, and one of R¹, R² and R³ is —P(═O)(OH)—O—CH₂CH₂—NH—C(O)—(CH₂)₆—N₃.

Surfactants are surface-active compounds, such as anionic, cationic, nonionic and amphoteric (zwitterionic) surfactants, block polymers, polyelectrolytes, and mixtures thereof.

Anionic surfactants are for example alkali, alkaline earth or ammonium salts of sulfonates, sulfates, phosphates, carboxylates, and mixtures thereof. Examples of sulfonates are alkylarylsulfonates, diphenylsulfonates, alpha-olefin sulfonates, lignine sulfonates, sulfonates of fatty acids and oils, sulfonates of ethoxylated alkylphenols, sulfonates of alkoxylated arylphenols, sulfonates of condensed naphthalenes, sulfonates of dodecyl- and tridecylbenzenes, sulfonates of naphthalenes and alkylnaphthalenes, sulfosuccinates or sulfosuccinamates. Examples of sulfates are sulfates of fatty acids and oils, of ethoxylated alkylphenols, of alcohols, of ethoxylated alcohols, or of fatty acid esters. Examples of phosphates are phosphate esters. Examples of carboxylates are alkyl carboxylates, and carboxylated alcohol or alkylphenol ethoxylates.

Cationic surfactants are for example quaternary surfactants, for example quaternary ammonium compounds with one or two hydrophobic groups, or salts of long-chain primary amines. Suitable amphoteric surfactants are alkylbetains and imidazolines. Suitable block polymers are block polymers of the A-B or A-B-A type comprising blocks of polyethylene oxide and polypropylene oxide, or of the A-B—C type comprising alkanol, polyethylene oxide and polypropylene oxide. Suitable polyelectrolytes are polyacids or polybases. Examples of polyacids are alkali salts of polyacrylic acid or polyacid comb polymers. Examples of polybases are polyvinylamines or polyethyleneamines.

Suitable non-ionic surfactants are for example alkoxylate surfactants, N-substituted fatty acid amides, amine oxides, esters, sugar-based surfactants, polymeric surfactants, and mixtures thereof. Examples of alkoxylate surfactants are compounds such as alcohols, alkylphenols, amines, amides, arylphenols, fatty acids or fatty acid esters which have been alkoxylated with 1 to 50 equivalents of an alkylene oxide. Ethylene oxide and/or propylene oxide may be employed for the alkoxylation, preferably ethylene oxide. Examples of N-substituted fatty acid amides are fatty acid glucamides or fatty acid alkanolamides. Examples of esters are fatty acid esters, glycerol esters or monoglycerides. Examples of sugar-based surfactants are sorbitans, ethoxylated sorbitans, sucrose and glucose esters or alkylpolyglucosides. Examples of polymeric surfactants are homo- or copolymers of vinylpyrrolidone, vinylalcohols, or vinylacetate.

Amphoteric surfactants are compounds with a cationic and an anionic group. The cationic group is generally an ammonium group and the anionic group is generally selected from the group consisting of oxy (O⁻), carboxylate, sulfonate and phosphonate groups, the terms carboxylate, sulfonate and phosphate denoting here anions (not esters). Examples are taurin (2-aminoethanesulfonic acid), the phosphatidyl cholines, cocamidopropyl betaine, cocoamidopropyl hydroxysultaine, acyl ethylenediamines and N-alkyl amino acids.

The surfactant is preferably selected from the group consisting of non-ionic surfactants, zwitterionic surfactants and mixtures thereof.

Preferably, the non-ionic surfactants are selected from polyalkyleneglycolethers. The polyalkyleneglycolethers are in turn preferably selected from the group consisting of polyoxyethylenecetylstearylethers having from 5 to 50 oxyethylene repeating units and polyoxyethylene-(optionally hydrogenated) castor oil ethers having from 5 to 50 oxyethylene repeating units. A particularly useful surfactant is Cremophor® ELP, the product obtained from reacting castor oil with ethylene oxide in a molar ratio of 1:35.

Preferable amphoteric/zwitterionic surfactants are selected from compounds with a quaternary ammonium group and a phosphate group. In particular, the cationic surfactant is a phosphatidylcholine, e.g. phosphatidylcholines in which the fatty acid residues in the glyceride moiety are C₁₂-C₂₀-fatty acid residues, such as lauroyl, myristoyl, palmitoyl, stearinoyl or arachinoyl or unsaturated radicals, like radicals derived from oleic acid or palmitoleic acid.

Substances which are suitable to link the lipid and the albumin are compounds which contain a carboxyl group, a sulfonic acid group or an active ester group (for the reaction with the amino groups of the albumin) and at least one further functional group suitable for the reaction with the functional group of the functionalized lipid. If, for example, the functional group of the functionalized lipid is an azido group, the substance suitable to link the lipid and the albumin suitably contains an azide-reactive group, such as C—C-triple bond, especially a strained C—C-triple bond. A specific example for such a compound is a sulfo-dibenzoyl-cyclooctyne-N-hydroxysuccinimide compound, e.g. of following formula:

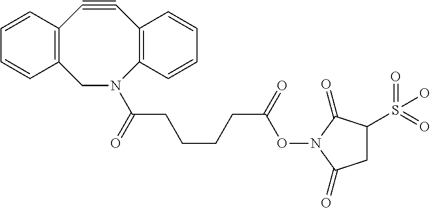 [SMILES: O=C(CCCCC(=O)N1Cc2ccccc2C#Cc2ccccc21)ON1C(=O)CC(S(=O)(=O)O)C1=O]

Here, the carbonyl-(sulfo-N-oxysuccinimide) group is an active ester group which allows subsequent amidation with amino groups of the albumin.

Another specific example for such a compound is the compound of following formula:

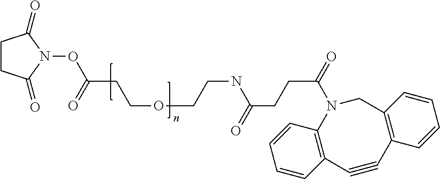

Here, too, the carbonyl-N-oxysuccinimide group is an active ester group which allows subsequent amidation with amino groups of the albumin.

If, for example, the functional group of the functionalized lipid is a hydroxy, primary or secondary amino or thiol group, the substance suitable to link the lipid and the albumin can be a dicarboxylic acid (such as oxalic acid, malonic acid, succinic acid, adipic acid, maleic acid, fumaric acid etc.) or a compound with a carboxylic acid and a sulfonic acid group or a compound with a sulfonate group (as leaving group) and a carboxylic acid group. If, for example, the functional group of the functionalized lipid is a carboxylic acid and a sulfonic acid group, the substance suitable to link the lipid and the albumin can be a carboxylic acid or sulfonic acid carrying additionally a hydroxy, primary or secondary amino or thiol group, such as 4-hydroxybutyric acid, 4-aminobutyric acid, 4-mercaptobutyric acid and the like. If, for example, the functional group of the functionalized lipid is a C—C double bond, the substance suitable to link the lipid and the albumin can be a carboxylic acid or sulfonic acid carrying additionally a tetrazine moiety or a thiol group (especially if the C—C double bond on the functionalized lipid is bound to a carbonyl group) or two conjugated C—C double bonds. If, for example, the functional group of the functionalized lipid is a triple bond, the substance suitable to link the lipid and the albumin can be a carboxylic acid or sulfonic acid carrying additionally an azide group.

As can be seen, a plethora of organic reactions and thus of substances which are suitable to link the lipid and the albumin are suitable.

In a specific embodiment, the substance which is suitable to link the lipid and the albumin is sulfo-dibenzoyl-cyclooctyne-N-hydroxysuccinimide (DBCO).

The organic solvent is preferably selected from the group consisting of aliphatic hydrocarbons, such as pentane, hexane or heptane, chlorinated alkanes, such as dichloromethane, trichloromethane or dichloroethane, cycloaliphatic hydrocarbons, such as cyclohexane, dialkylethers, such as diethylether, methyl-tert-butyl ether or methyl-isobutyl ether, cyclic ethers, such as tetrahydrofuran or the dioxanes, aliphatic carboxylic acid esters, such as ethylacetate or ethylpropionate, alkylnitrils, such as acetonitril, dimethylformamid, dimethylacetamid, and dimethylsufoxid, and is in particular an aliphatic carboxylic acid ester, specifically ethylacetate.

The solution of the cargo substance in water contains the cargo substance in an overall amount of preferably up to 200 g per 1 of the solution.

Preferably, the weight ratio of the water-in-oil emulsion obtained in step (a.2) and the aqueous phase to which the former is transferred in step (a.3) is of from 1:10 to 1:1000.

Preferably, the water-in-oil emulsion obtained in step (a.2) is transferred in step (a.3) to the aqueous phase via an orifice, in particular via a syringe needle, of a diameter of at most 1400 μm, e.g. of at most 1000 μm or at most 500 μm (the diameter being the inner diameter).

The nanoparticles formed in the double emulsion of step (a.3) can be freed from undesired large by-products before further reaction, e.g. by filtration through a filter with a suitable pore size. If desired, the nanoparticles can then be concentrated, e.g. by centrifugation and subsequent removal of the supernatant, of by filtration with small pore size.

In this variant of the method of the invention, step (b) is included in steps (a.1) to (a.3).

Suitable steps (c) which follow are steps (c.3), (c.6) or (c.7). Specifically, step (c.3) follows.

Suitable linking groups have already been described above as substances which are suitable to link the lipid and the albumin. As said, they are derived from compounds which contain a carboxyl group, a sulfonic acid group or an active ester group (for the reaction with the amino groups of the albumin) and at least one further functional group suitable for the reaction with the functional group of the functionalized lipid. If, for example, the functional group of the functionalized lipid is an azido group, the substance suitable to link the lipid and the albumin suitably contains an azide-reactive group, such as C—C-triple bond, especially a strained C—C-triple bond.

A specific example for such a compound is a sulfo-dibenzoyl-cyclooctyne-N-hydroxysuccinimide compound of the following formula

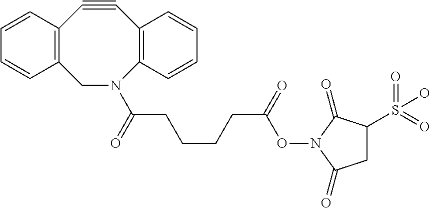 [SMILES: O=C(CCCCC(=O)N1Cc2ccccc2C#Cc2ccccc21)ON1C(=O)CC(S(=O)(=O)O)C1=O]

Here, the carbonyl-(sulfo-N-oxysuccinimide) group is an active ester group which allows amidation with amino groups of the albumin under very mild conditions (room temperature; water as solvent, pH around 7). The SO₃ group can either be present as sulfonic acid group —S(O)₂OH or as a sulfonate, e.g. as sodium sulfonate (—S(O)₂ONa), the latter leading to a better solubility of the compound in aqueous medium.

Another specific example for such a compound is the dibenzoyl-cyclooctyne compound of following formula, also containing a carbonyl-N-oxysuccinimide group as active ester group.

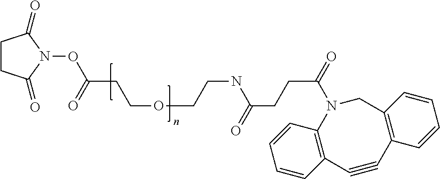

To obtain the linking group to which the albumin is already attached, the albumin and the substance which is suitable to link the lipid and the albumin, e.g. the above (sulfo-) dibenzoyl-cyclooctyne-N-oxysuccinimide compound, are reacted with each other. As said, given the active ester moiety in the sulfo-dibenzoyl-cyclooctyne-N-oxysuccinimide, the amino groups of albumin readily substitute the N-oxysuccinimide at the carbonyl group and form amide bonds to give an albumin-linking group substance, as sketched here exemplary for the first dibenzoyl-cyclooctyne compound:

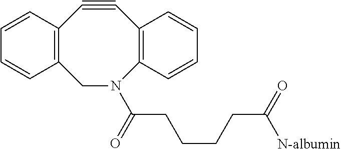

In step (c.3), such albumin-linking group substances are reacted with the nanoparticle of step (a.3). In case of the specific albumin-linking group substance shown above and the specific azido-phosphatidyl-modified lipid described above, the azido group of the lipid reacts readily with the strained triple bond in the above-depicted specific albumin-linking group substance under mild conditions (room temperature; water as solvent, pH around 7) in a [2+3] reaction to a triazole, thus covalently connecting the albumin to the lipid material of the nanoparticle.

Step (c.3) is then followed by step (d.2.1), which is either followed by step (e.1.1) and then (e.2), or by step (e.1.2); or step (c.3) is followed by step (d.2.2).

Specifically, following reaction suit is carried out: (d.2.1)→(e.1.2).

Specifically, in step (d.2.1) the albumin of the substance obtained in step (c.3) is reacted with only a part of the linker. Since the linker contains preferably a polyethyleneglycol chain, the part of the linker to be connected is suitably a polyethyleneglycol chain carrying on one terminus a functional group suitable to react with the amino groups of the albumin, i.e. preferably a carboxyl, sulfonic acid or active ester group, and on the other terminus a functional group suitable to react with the rest of the linker. Suitable couples of functional groups on the two linking group parts are those listed above for reacting the functionalized lipid with the substance suitable to link the lipid and the albumin. Specifically, a combination of azide/strained C—C triple bond is used.

The linking group part to be reacted with the albumin is specifically a compound of following formula:

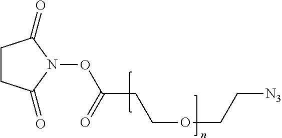

where n is from 2 to 498 and is very specifically 4. The carbonyl-(sulfo-N-oxysuccinimide) group is an active ester group which allows amidation with amino groups of the albumin under very mild conditions (room temperature; water as solvent, pH around 7).

The suitable part of the linking group to be attached in step (e.1.2) is for this specific case for example a substance of following formula:

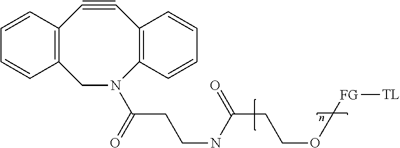

where n is from 2 to 498, where the two n's of the two linking parts are in sum 10 to 500; and FG is a functional group via which the targeting ligand TL is attached, in particular a carboxamide group —C(O)—NH— if the targeting ligand is a peptide or a protein or generally a substance with primary or secondary amino groups. In case of polyoxyalkylene-containing polymers as targeting ligands TG, the group FG is suitably an ester group —C(O)—O—.

The second part of the linker is bound to the targeting linker under conditions analogous to those described above for the reaction between albumin and linking group.

Like in the above-described reaction, the azido group of the first part of the specific linker readily reacts with the strained triple bond in the above-depicted specific second part of the linker under mild conditions (room temperature; water as solvent, pH around 7) in a [2+3] reaction to a triazole, thus covalently connecting the targeting ligand and the albumin via a linker.

If the method is to be carried out via other suits of the above-described steps, the reactions can all be carried out in analogy to the specific reaction suit described above. For instance, all reactions which involve a coupling between albumin and another compound or targeting ligand containing amino groups and another compound can be carried out in analogy to the reactions described above for the reaction between albumin and linking group A. Such other compounds have to carry a group which is reactive towards the amino groups of the albumin or the targeting ligand, such as a carboxyl group or a sulfonic acid group or an active ester group, and have to carry a further functional group to react with those parts which are still to be attached.

In another aspect, the invention relates to a nanoparticle obtainable by the method of the invention.

### Pharmaceutical Composition

The invention also relates to a pharmaceutical composition containing a plurality of nanoparticles of the invention and a pharmaceutically acceptable carrier.

The nanoparticles of the present invention can be provided in the form of a pharmaceutical composition comprising a plurality of nanoparticles as described herein, and a pharmaceutically acceptable carrier. The carrier is chosen to be suitable for the intended way of administration which can be, for example, peroral or parenteral administration, e.g. intravascular, subcutaneous or, most commonly, intravenous injection, transdermal application, or topical applications such as onto the skin, nasal or buccal mucosa or the conjunctiva.

The nanoparticles of the invention can be provided in the form of liquid pharmaceutical compositions. These compositions typically comprise a carrier selected from aqueous solutions which may comprise one or more than one water-soluble salt and/or one or more than one water-soluble polymer. If the composition is to be administered by injection, the carrier is typically an isotonic aqueous solution (e.g. a solution containing 150 mM NaCl, 5 wt-% dextrose or both). Such carrier also typically has an appropriate (physiological) pH in the range of 7.3-7.4.

Alternatively, the nanoparticles of the invention can be provided in the form of solid or semisolid pharmaceutical compositions, e.g. for peroral administration or as a depot implant. Suitable carrier for these compositions include, but are not limited to, pharmaceutically acceptable polymers selected from the group consisting of homopolymers and copolymers of N-vinyl lactams (especially homopolymers and copolymers of N-vinyl pyrrolidone, e.g. polyvinylpyrrolidone, copolymers of N-vinyl pyrrolidone and vinyl acetate or vinyl propionate), cellulose esters and cellulose ethers (in particular methylcellulose and ethylcellulose, hydroxyalkylcelluloses, in particular hydroxypropylcellulose, hydroxyl-alkylalkylcelluloses, in particular hydroxypropylmethylcellulose, cellulose phthalates or succinates, in particular cellulose acetate phthalate and hydroxypropylmethylcellulose phthalate, hydroxypropylmethylcellulose succinate or hydroxypropylmethylcellulose acetate succinate), high molecular weight polyalkylene oxides (such as polyethylene oxide and polypropylene oxide and copolymers of ethylene oxide and propylene oxide), polyvinyl alcohol-polyethylene glycol-graft copolymers, polyacrylates and polymethacrylates (such as methacrylic acid/ethyl acrylate copolymers, methacrylic acid/methyl methacrylate copolymers, butyl methacrylate/2-dimethylaminoethyl methacrylate copolymers, poly(hydroxyalkyl acrylates), poly(hydroxyalkyl methacrylates)), polyacrylamides, vinyl acetate polymers (such as copolymers of vinyl acetate and crotonic acid, partially hydrolyzed polyvinyl acetate), polyvinyl alcohol, oligo- and polysaccharides such as carrageenans, galactomannans and xanthan gum, alginate, acacia gum, gelatin or mixtures of one or more thereof. Solid carrier ingredients may be dissolved or suspended in a liquid suspension of nanoparticles of the invention and the liquid suspension medium may be, at least partially, removed.

Freeze-dried nanoparticle preparations are particularly suitable for preparing solid or semisolid pharmaceutical compositions and dosage forms of nanoparticles of the invention. Suitable methods for freeze-drying of nanoparticles are known in the art and may include the use of cryoprotectants (e.g. trehalose, sucrose, sugar alcohols such as mannitol, surface active agents such as the polysorbates, poloxamers, glycerol and/or dimethylsulfoxide). Solid dosage forms of nanoparticles of the invention which are particularly suitable for peroral administration include, but are not limited to, capsules (e.g. hard or soft gelatin capsules), tablets, pills, powders and granules, which may optionally be coated. Coatings of peroral solid dosage forms intended for delivering the nanoparticles to particular regions within the intestine (such as to inflamed intestinal regions of patients suffering from inflammatory bowel diseases) are expediently gastro-resistant.

### Medical Use

The invention relates moreover to the nanoparticles of the invention for use as a medicament; and to nanoparticles of the invention for use in the treatment or prophylaxis of conditions, disorders or deficits of the central nervous system (CNS); liver, inflammatory diseases, hyperproliferative diseases, a hypoxia-related pathology and a disease characterized by excessive vascularization.

CNS disorders are for example schizophrenia, depression, motivation disturbances, bipolar disorders, cognitive dysfunctions, in particular cognitive dysfunctions associated with schizophrenia, cognitive dysfunctions associated with dementia (Alzheimer's disease), Parkinson's disease, anxiety, dyskinesia, in particular L-DOPA induced dyskinesia (LID), especially dyskinesia associated with L-DOPA therapy to treat Parkinson's disease, substance-related disorders, especially substance use disorder, substance tolerance conditions associated with substance withdrawal, attention deficit disorders with or without hyperactivity, eating disorders, and personality disorder as well as pain.

Inflammatory diseases are for example atherosclerosis, rheumatoid arthritis, asthma, inflammatory bowel disease, psoriasis, in particular psoriasis vulgaris, psoriasis capitis, psoriasis guttata, psoriasis inversa; neurodermatitis; ichtyosis; alopecia areata; alopecia totalis; alopecia subtotalis; alopecia universalis; alopecia diffusa; atopic dermatitis; lupus erythematodes of the skin; dermatomyositis of the skin; atopic eczema; morphea; scleroderma; alopecia areata Ophiasis type; androgenic alopecia; allergic dermatitis; irritative contact dermatitis; contact dermatitis; pemphigus vulgaris; pemphigus foliaceus; pemphigus vegetans; scarring mucous membrane pemphigoid; bullous pemphigoid; mucous membrane pemphigoid; dermatitis; dermatitis herpetiformis Duhring; urticaria; necrobiosis lipoidica; erythema nodosum; prurigo simplex; prurigo nodularis; prurigo acuta; linear IgA dermatosis; polymorphic light dermatosis; erythema solaris; exanthema of the skin; drug exanthema; purpura chronica progressiva; dihydrotic eczema; eczema; fixed drug exanthema; photoallergic skin reaction; and perioral dermatitis.

Hyperproliferative diseases are for example a tumor or cancer disease, precancerosis, dysplasia, histiocytosis, a vascular proliferative disease and a virus-induced proliferative disease. In particular, the hyperproliferative disease is a tumor or cancer disease selected from the group consisting of diffuse large B-cell lymphoma (DLBCL), T-cell lymphomas or leukemias, e.g., cutaneous T-cell lymphoma (CTCL), noncutaneous peripheral T-cell lymphoma, lymphoma associated with human T-cell lymphotrophic virus (HTLV), adult T-cell leukemia/lymphoma (ATLL), as well as acute lymphocytic leukemia, acute nonlymphocytic leukemia, acute myeloid leukemia, chronic lymphocytic leukemia, chronic myelogenous leukemia, Hodgkin's disease, non-Hodgkin's lymphoma, myeloma, multiple myeloma, mesothelioma, childhood solid tumors, glioma, bone cancer and soft-tissue sarcomas, common solid tumors of adults such as head and neck cancers (e.g., oral, laryngeal and esophageal), genitourinary cancers (e.g., prostate, bladder, renal (in particular malignant renal cell carcinoma (RCC)), uterine, ovarian, testicular, rectal, and colon), lung cancer (e.g., small cell carcinoma and non-small cell lung carcinoma, including squamous cell carcinoma and adenocarcinoma), breast cancer, pancreatic cancer, melanoma and other skin cancers, basal cell carcinoma, metastatic skin carcinoma, squamous cell carcinoma of both ulcerating and papillary type, stomach cancer, brain cancer, liver cancer, adrenal cancer, kidney cancer, thyroid cancer, medullary carcinoma, osteosarcoma, soft-tissue sarcoma, Ewing's sarcoma, veticulum cell sarcoma, and Kaposi's sarcoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, leiomyosarcoma, rhabdomyosarcoma, squamous cell carcinoma, adenocarcinoma, sweat gland carcinoma, sebaceous gland carcinoma, papillary carcinoma, glioblastoma, papillary adenocarcinomas, cystadenocarcinoma, bronchogenic carcinoma, seminoma, embryonal carcinoma, Wilms' tumor, small cell lung carcinoma, epithelial carcinoma, astrocytoma, medulloblastoma, craniopharyngioma, ependymoma, pinealoma, hemangioblastoma, acoustic neuroma, oligodendroglioma, meningioma, neuroblastoma, retinoblastoma, glaucoma, hemangioma, heavy chain disease and metastases.

The precancerosis are for example selected from the group consisting actinic keratosis, cutaneaous horn, actinic cheilitis, tar keratosis, arsenic keratosis, x-ray keratosis, Bowen's disease, bowenoid papulosis, lentigo maligna, lichen sclerosus, and lichen rubber mucosae; precancerosis of the digestive tract, in particular erythroplakia, leukoplakia, Barrett's esophagus, Plummer-Vinson syndrome, crural ulcer, gastropathia hypertrophica gigantea, borderline carcinoma, neoplastic intestinal polyp, rectal polyp, porcelain gallbladder; gynaecological precancerosis, in particular carcinoma ductale in situ (CDIS), cervical intraepithelial neoplasia (CIN), endometrial hyperplasia (grade III), vulvar dystrophy, vulvar intraepithelial neoplasia (VIN), hydatidiform mole; urologic precancerosis, in particular bladder papillomatosis, Queyrat's erythroplasia, testicular intraepithelial neoplasia (TIN), carcinoma in situ (CIS); precancerosis caused by chronic inflammation, in particular pyoderma, osteomyelitis, acne conglobata, lupus vulgaris, and fistula.

Dysplasia is frequently a forerunner of cancer, and is can be found in e.g. the epithelia; it is the most disorderly form of non-neoplastic cell growth, involving a loss in individual cell uniformity and in the architectural orientation of cells. Dysplastic cells often have abnormally large, deeply stained nuclei, and exhibit pleomorphism. Dysplasia characteristically occurs where there exists chronic irritation or inflammation. Dysplastic disorders which can be treated with the compounds of the present invention include, but are not limited to, anhidrotic ectodermal dysplasia, anterofacial dysplasia, asphyxiating thoracic dysplasia, atriodigital dysplasia, bronchopulmonary dysplasia, cerebral dysplasia, cervical dysplasia, chondroectodermal dysplasia, cleidocranial dysplasia, congenital ectodermal dysplasia, craniodiaphysial dysplasia, craniocarpotarsal dysplasia, craniometaphysial dysplasia, dentin dysplasia, diaphysial dysplasia, ectodermal dysplasia, enamel dysplasia, encephalo-ophthalmic dysplasia, dysplasia epiphysialis heminelia, dysplasia epiphysialis multiplex, dysplasia epiphysalis punctata, epithelial dysplasia, faciodigitogenital dysplasia, familial fibrous dysplasia of jaws, familial white folded dysplasia, fibromuscular dysplasia, fibrous dysplasia of bone, florid osseous dysplasia, hereditary renal-retinal dysplasia, hidrotic ectodermal dysplasia, hypohidrotic ectodermal dysplasia, lymphopenic thymic dysplasia, mammary dysplasia, mandibulofacial dysplasia, metaphysical dysplasia, Mondini dysplasia, monostotic fibrous dysplasia, mucoepithelial dysplasia, multiple epiphysial dysplasia, oculoauriculovertebral dysplasia, oculodentodigital dysplasia, oculovertebral dysplasia, odontogenic dysplasia, ophthalmomandibulomelic dysplasia, periapical cemental dysplasia, polyostotic fibrous dysplasia, pseudoachondroplastic spondyloepiphysial dysplasia, retinal dysplasia, septooptic dysplasia, spondyloepiphysial dysplasia, and ventriculoradial dysplasia.

A hypoxia related pathology is for example diabetic retinopathy, ischemic reperfusion injury, ischemic myocardial and limb disease, ischemic stroke, sepsis and septic shock (see, e.g. Liu F Q, et al., Exp Cell Res. 2008 Apr. 1; 314(6):1327-36).

A disease characterized by pathophysiological hyper-vascularization is for example angiogenesis in osteosarcoma (see, e.g.: Yang, Qing-cheng et al., Dier Junyi Daxue Xuebao (2008), 29(5), 504-508), macular degeneration, in particular, age-related macular degeneration and vasoproliferative retinopathy (see e.g. Kim J H, et al., J Cell Mol Med. 2008 Jan. 19).

### Method

The above-described steps (a.1), (a.2) and (a.3) offer a very useful approach to nanoparticles of a cargo substance which is stable in water and which is surrounded by or embedded in a lipid which avoid tedious and energy-consuming process steps used in the prior art, such as various sonication and phase separation steps. They are not only applicable in the production of nanoparticles of the invention containing an albumin corona and a targeting ligand, but also to simpler cargo/lipid nanoparticles containing just a cargo substance which is stable in water and which is surrounded by or embedded in a lipid. Thus, in another aspect, the invention also relates to a method for producing nanoparticles in which a cargo substance which is stable in aqueous solution is embedded in or surrounded by a lipid material, comprising
(1) dissolving in an organic solvent the lipid material, one or more surfactants and optionally one or more substances which under the given conditions are suitable to provide the lipid material with anchoring groups for further reactions;
(2) mixing the solution obtained in step (1) with a solution of the cargo substance in water to give a water-in-oil emulsion; and
(3) transferring the water-in-oil emulsion obtained in step (2) to an aqueous phase to give an oil-in-water emulsion.

Details given above to steps (a.1) to (a.3) apply here analogously. The one or more substances which under the given conditions are suitable to provide the lipid material with anchoring groups for further reactions are for example the functionalized lipids described above.

If desired, step (3) can be followed by steps corresponding to those described above under (c), (d) and (e).

The nanoparticles of the invention show a good uptake into the targeted cells. Simultaneously, they avoid the problems associated with the uncontrolled formation of a protein corona when introduced into a biological medium, such as blood, and thus show a reduced clearance rate from blood circulation and no or only low undesired cytotoxicity. Moreover, they are able to cross the blood/brain barrier.

The invention is now illustrated by the following figures and examples.

### EXAMPLES

### Abbreviations

Cremophor® ELP Nonionic solubilizer made by reacting castor oil with ethylene oxide in a molar ratio of 1:35, followed by a purification process, from BASF SE
Lipoid S-100 highly purified phosphatidyl choline from soy beans comprising at least 94% phosphatidyl choline from Lipoid GmbH, Germany
DBCO dibenzocyclooctyne
FACS fluorescence activated cell sorting
NIR near-infrared dye for in vivo imaging (Vivotag 680 XL)
PEG polyethylene glycol (also for the polyethylene glycol radical or diradical)
RT room temperature (20-25° C.)

### FIGURES

FIG. 1: FACS analysis of human cerebral microvascular endothelial cells (hCMEC/D3) incubated for 90 min with solid lipid nanoparticles (SLNPs) having fluorescent cargo and different surface structures. The structural composition of the individual SLNPs tested for cellular uptake is depicted on the left, whereas the distribution of fluorescence intensity per cell count is given on the right.

FIG. 2: Background- and live cells-corrected readout of the FACS analysis depicted in FIG. 1. The cellular uptake is given as % values of fluorescent dye-positive living cells (upper part of FIG. 2). The structural composition of the individual SLNPs tested for cellular uptake is depicted on the lower part of FIG. 2.

FIG. 3: Comparison of NIR fluorescence in mouse 1001 dosed with NIR-labeled IgG-loaded SLNP-HSA-PEG nanoparticles with that in a naïve animal dosed with placebo (included as control for determination of background (autofluorescence) levels). The distribution of fluorescence in the mice was followed over a time course of 48 h after injection of the sample into the tail vein. The fluorescence in the naïve mouse at 4 h marked with an arrow resulted from transfer of material during wake phases due to group housing with the nanoparticle-dosed animal.

FIG. 4: Comparison of NIR fluorescence in mouse 1001 dosed with NIR-labeled IgG-loaded SLNP-HSA-PEG-Tf nanoparticles in mouse 2001 with that in a naïve animal dosed with placebo (included as control for determination of background (autofluorescence) levels). The distribution was followed over a time course of 48 h after injection into the tail vein. The fluorescence in the naïve mouse at 4 h marked with an arrow resulted from transfer of material during wake phases due to group housing with the dosed animal.

FIG. 5: Images of tissue samples from the endothelium of the mouse brain cortex of an animal treated (tail vain injection) with free human IgG (upper image) and, as comparison, of an animal treated with human IgG-loaded SLNP-HSA-PEG-Tf (lower image). The samples were stained with anti-human HSA antibody. The arrows mark human HSA-specific staining at the brain cortex vasculature indicating that human IgG-loaded SLNP-HSA-PEG-Tf was bound to the brain cortex endothelium. Tissue samples were taken 24 h after the tail vain injections.

FIG. 6: Images of tissue samples from the mouse brain cerebellum of an animal treated (tail vain injection) with free human IgG (upper image) and, as comparison, of an animal treated with human IgG-loaded SLNP-HSA-PEG-Tf (lower image). The samples were stained with anti-human IgG antibody. The arrows indicate human IgG-specific staining at Purkinje-cells indicating the presence of human IgG delivered by human IgG-loaded SLNP-HSA-PEG-Tf. Tissue samples were taken 24 h after the tail vain injections.

### EXAMPLES

### I. Production of Solid Lipid Nanoparticles

Solid lipid nanoparticles (SLNPs) were produced from stocks of surfactants and lipids. Cremophor® ELP was dissolved at 100 mg/mL in ethyl acetate. 100% phosphatidyl choline from soy beans (Lipoid S-100) was dissolved at 100 mg/mL in ethyl acetate. 16:0 azidocaproyl phosphatidyl ethanolamine was dissolved in ethyl acetate at 4 mg/mL. Trilaurin was melted at 60° C. A water based solution containing an antibody (human IgG) as active pharmaceutical ingredient (API) was prepared for encapsulation into SLNPs. 111.1 μL Cremophor® ELP, 222.2 μL phosphatidyl choline, 4 μL azidocaproyl phosphatidyl ethanolamine and 74.1 μL melted Trilaurin were mixed in a 1.5 mL microcentrifuge tube. Ethyl acetate was partly evaporated under a constant stream of nitrogen gas until the solution become slightly viscous.

10 μL of the water-based phase containing the API were added to the microcentrifuge tube and the mixture was agitated until the solution was uniform and transparent. If necessary, the mixture was heated to 45° C. until the solution was uniform and transparent. The solution was transferred to an appropriately-sized glass syringe pre-warmed to 60° C. A fine 31G cannula was pre-warmed to 60° C. and attached to the syringe. The syringe content was slowly injected at 400 μL/min into water containing 0.0001% (w/w) of the surfactant Poloxamer P188 under stirring at 600 rpm.

The produced SLNPs were filtered through a 0.45 μm pore size modified PES filter to remove unwanted large by-products. SLNPs were concentrated by using hollow fiber filters for tangential flow filtration with a molecular weight cut-off of 300 kDa.

If required for detection in in vivo imaging studies, the API (human IgG) was labeled with a fluorescent marker (e.g. Vivotag 680 XL-N-hydroxysuccinimidyl ester) with a protein:dye ratio of approximately 1:1 prior to encapsulation into SLNPs.

### II. Production of Surface Modified Solid Lipid Nanoparticles

140.4 mg of human serum albumin (HSA) were modified by attaching 3.35 mg dibenzocyclooctyne-sulfo-N-hydroxysuccinimidyl ester of formula

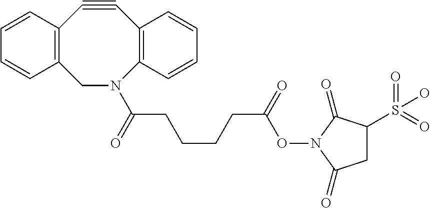 [SMILES: O=C(CCCCC(=O)N1Cc2ccccc2C#Cc2ccccc21)ON1C(=O)CC(S(=O)(=O)O)C1=O]

or 4.09 mg dibenzocyclooctyne-PEG₄-N-hydroxysuccinimidyl ester of formula

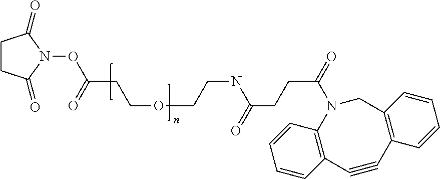

where n=4

in 10 mL of a 50 mM phosphate buffer to the protein surface of HSA at pH 7.4 for ≥12 h at room temperature (RT). If required, an amine-reactive fluorescent marker (e.g. 3.89 mg Dylight 650—N-hydroxysuccinimidyl ester) was added at this step to produce a fluorescently labeled albumin for detection of final nanoparticles. Unbound reactants were removed by ultrafiltration/diafiltration using spin columns equipped with PES filter membranes having a molecular weight cut-off of ≤50 kDa. The modified albumin (HSA-DBCO) was concentrated by filtration and added in excess to the SLNP solution. The mixture was incubated for ≥12 h at RT to let the dibenzocyclooctyne on the HSA-DBCO react with the azide group of the azidocaproyl phosphatidyl ethanolamine on the SLNPs. Unbound HSA-DBCO was removed by using hollow fiber filters for tangential flow filtration with a molecular weight cut-off of 300 kDa. Then, new azide groups were introduced on the resulting SLNPs with albumin corona (SLNP-HSA) by adding 8.14 mg azide-PEG4-N-hydroxysuccinimidyl ester of following formula:

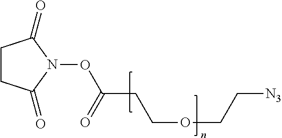

where n is 4
and incubating for ≥2 h at RT. Unbound azide-PEG4-N-hydroxysuccinimidyl ester was removed by using hollow fiber filters for tangential flow filtration with a molecular weight cut-off of 300 kDa.

The targeting ligand was modified by attaching dibenzocyclooctyne-PEG-N-hydroxysuccinimidyl ester with a molecular weight of 3.4 kDa of the formula

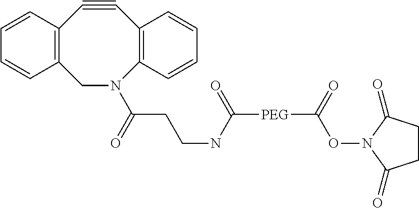

where PEG is a polyethyleneglycol chain with 3.4 kDa, to the surface of the respective targeting ligand. In case of transferrin as targeting ligand, 161.5 mg protein were incubated with 25.15 mg dibenzocyclooctyne-PEG-N-hydroxysuccinimidyl ester in 10 mL of a 50 mM phosphate buffer at pH 7.4 for ≥12 h at RT. Unbound dibenzocyclooctyne-PEG-N-hydroxysuccinimidyl ester was removed by ultrafiltration/diafiltration using spin columns equipped with PES filter membranes having a molecular weight cutoff of ≤50 kDa. The modified Transferrin (Tf-PEG-DBCO) was concentrated and added to SLNP-HSA in excess. The mixture was incubated for ≥12 h at RT. Unbound Tf-PEG-DBCO was removed by using hollow fiber filters for tangential flow filtration with a molecular weight cut-off of 300 kDa. The purified nanoparticles with albumin corona and Transferrin linked to the albumin corona (SLNP-HSA-PEG-Tf) were further concentrated and diafiltered against a suitable buffer for subsequent use as needed using hollow fiber filters for tangential flow filtration with a molecular weight cut-off of 300 kDa. The nanoparticle solution was sterile filtered using a PES filter of 0.45 μm pore size if intended for use in animals.

Other surface modifications were produced in analogy to the method described above. Nanoparticles with direct immobilization of targeting ligands like transferrin (SLNP-PEG-Tf) were produced by omitting the conjugation of HSA and directly immobilizing Tf-PEG-DBCO to the azidocaproyl phosphatidyl ethanolamine on the nanoparticle surface. Nanoparticles with human serum albumin corona but without targeting function (SLNP-HSA) were produced by omitting further the conjugation steps after immobilization of HSA-DBCO. For the production of nanoparticles with a wheat germ agglutinin corona (SLNP-WGA) 23.5 mg WGA protein were conjugated with 1.69 mg dibenzocyclooctyne-sulfo-N-hydroxysuccinimidyl ester of following formula:

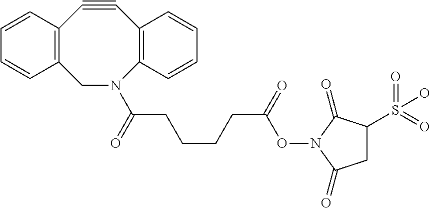 [SMILES: O=C(CCCCC(=O)N1Cc2ccccc2C#Cc2ccccc21)ON1C(=O)CC(S(=O)(=O)O)C1=O]

or 2.15 mg dibenzocyclooctyne-PEG4—N-hydroxysuccinimidyl ester of following formula:

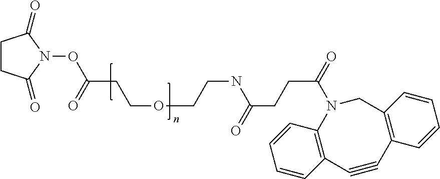

where n=4,
as described above for HSA. The resulting conjugate (WGA-DBCO) was immobilized on the surface of nanoparticles as described for HSA-DBCO.

### III. In Vitro Cellular Uptake Assays

Solid lipid nanoparticles (SLNPs) with fluorescent cargo and different surface structures were produced as described in example II. The fluorescent SLNPs were then tested in vitro for uptake into human cerebral microvascular endothelial cells (hCMEC/D3, hereinafter referred to as “D3 cells”) by using the following methodology.

D3 cells were grown in endothelial growth medium, comprising the supplements as listed in Table 1, using rat collagen-I coated (20 μg/cm²) 75 cm² cell culture flasks until they reached 90% confluency.

TABLE 1
Composition of EBM-2(G) growth medium used for
cultivation of human cerebral microvascular
endothelial cells (hCMEC/D3).

| | final |
|---|---|
| | concentration |
| component | in the medium |
| fetal calf serum (FCS) | 5% (v/v) |
| chemically defined lipid concentrate | 1% (v/v) |
| (CDLC ) | |
| HEPES | 10 mM |
| penicillin/streptomycin | 100 U/mL |
| P-mercaptoethanol | 50 μM |
| ascorbic acid | 5 μg/mL |
| basic Fibroblast Growth Factor (bFGF) | 1 ng/mL |
| hydrocortison | 1.4 μM |
| EBM-2 basal medium (Lonza) | ad |

The cells were detached with accutase for 10 min at 37° C. in the incubator (5% CO₂ and saturated humidity) and sub-cultivated with splitting rates of 1:3 to 1:5. For the cell uptake assay, D3 cells were seeded at 100,000 cells/cm² into rat collagen-I coated 12 well cell culture plates and cultivated for 2-3 more days in the incubator.

Stock solutions of fluorescent SLNPs were diluted in endothelial growth medium with 5% FCS and w/o chemically defined lipid concentrate (CDLC) to a final concentration of 400 μg/mL. The cell layers were washed once with pre-warmed PBS with Ca²⁺/Mg²⁺ and then incubated with 800 μL of SLNP-containing medium for 90 min inside the incubator. Thereafter, the incubated cells were harvested by washing the cell layers again twice with PBS w/o Ca²⁺/Mg²⁺ and treating the cells with trypsin for 5 min. Detached cells were collected in FACS cluster tubes and washed again with PBS w/o Ca²⁺/Mg²⁺.

Afterwards, the harvested cells were analyzed for uptake of fluorescent material via flow cytometry (FC). For this, the cell suspensions were stained for dead cells with Live/dead dye-eflour450 (eBioscience) for 1 h in the dark on ice. Cells were washed with PBS w/o Ca²⁺/Mg²⁺, spun down and resuspended in FC buffer (PBS w/o Ca²⁺/Mg²⁺ and 5% FCS). For flow cytometric acquisition a BD FACS Verse flow cytometer was used. Flow cytometric cell analysis was performed using the flowjo software on at least 50,000 live single cells per sample. Cellular uptake was defined as fluorescence-positive events of live single cells.

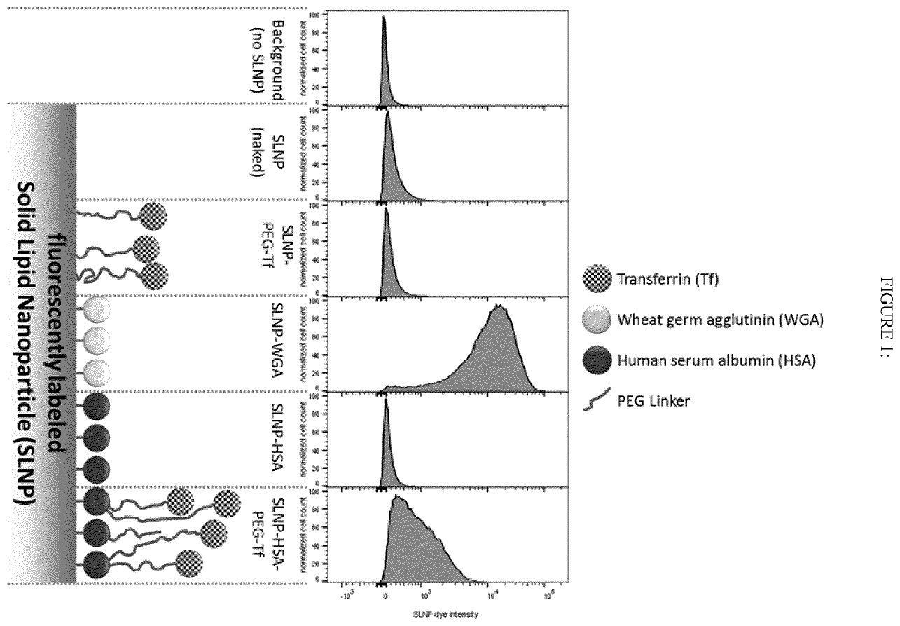

The results of the flow cytometric analysis are depicted in FIG. 1. Cells not treated with nanoparticles were used to determine background levels due to autofluorescence. As can be seen from FIG. 1, SLNPs without any surface modification (naked) or a single immobilized albumin corona (SLNP-HSA) show only background levels of fluorescence. As a positive control, wheat germ agglutinin (WGA) was immobilized on SLNPs. WGA is known to promiscuously bind to glycosylated proteins of the cell surface and trigger transient internalization (see for example Liu et al., Biomaterials, 2011, Vol. 32(30), pp. 7616-7624). Therefore, it is not useful for targeted delivery of nanoparticles but demonstrates the suitability of the assay and the maximum cellular uptake of SLNPs that can be achieved by unspecific receptor-mediated endocytosis. In contrast to this, the classical targeting approach via a known brain-targeting ligand (in this case Transferrin) attached to nanoparticles via a PEG linker (SLNP-PEG-Tf) fails in presence of 5% fetal calf serum. To enable cellular delivery to human brain cells using a brain-targeting ligand, SLNPs were covalently coated with a human serum albumin corona. The corona was further modified by attaching Transferrin via a PEG Linker (SLNP-HSA-PEG-Tf). A significant increase in cellular uptake of SLNP-HSA-PEG-Tf nanoparticles was observed over the classical targeting scheme implemented in the SLNP-PEG-Tf variant.

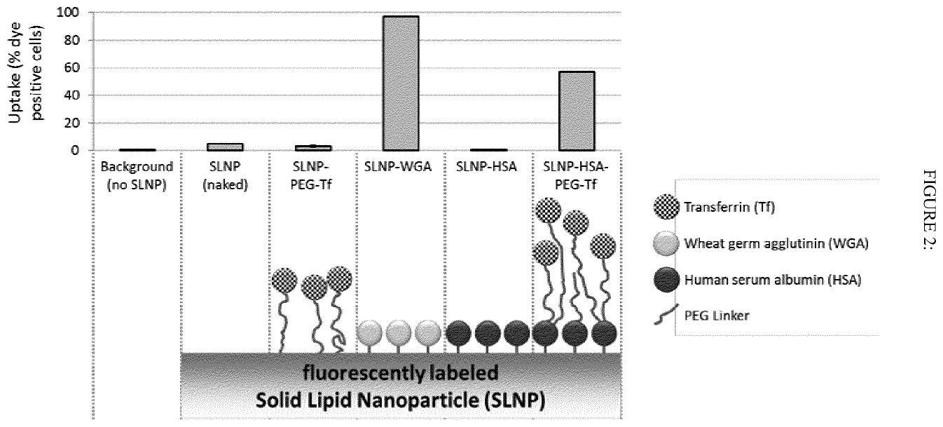

To better compare efficiency of targeting the different nanoparticle variants, the data from FIG. 1 was further analyzed and processed to take background fluorescence and live/dead cells into account. The amount of live cells showing uptake of fluorescent SLNPs above untreated cells (background) is given in FIG. 2. SLNP-WGA served as a positive control, as wheat germ agglutinin (WGA) is known to promiscuously bind to glycosylated proteins of the cell surface and trigger transient internalization (see for example Liu et al., Biomaterials, 2011, Vol. 32(30), pp. 7616-7624). Of the other samples, only SLNP-HSA-PEG-Tf showed significant uptake into the cells. The classical setup for targeting nanoparticles by having the targeting ligand immobilized directly on the surface via a PEG linker (as in SLNP-PEG-Tf) was not effective in the presence of 5% serum as were non-targeted particles as seen by SLNP (naked) and SLNP-HSA. This indicates that both the HSA corona and the targeting ligand Tf carried by said corona were required to effect substantial uptake by the cells.

### IV. In Vivo Distribution of SLNPs

To evaluate the in vivo uptake and distribution of the SLNPs, Male BALB/c mice (n=6 per group) were injected intravenously with placebo (naive), brain-targeted SLNPs (SLNP-HSA-PEG-Tf) or non-targeted SLNPs (SLNP-HSA-PEG). All injected SLNPs were loaded with a human IgG as cargo and the albumin corona was labeled with Vivotag 680 XL as near-infrared dye for in vivo imaging.

The evaluation of the in vivo uptake and distribution of the SLNPs was performed as follows. Per 20 g mouse 100 μL of a 100 mg/mL SLNP suspension were injected (=1 mg human IgG/mL=5 mg human IgG/kg body weight). As control, a subset of animals was injected with free human IgG labeled with VivoTag 680 XL (5 mg human IgG/kg body weight). To control for autofluorescence, another subset of animals was only injected with vehicle. Accordingly, animals were divided into 4 groups for near infrared fluorescence imaging (NIRF imaging):

Group 1—human IgG-loaded SLNPs labeled with VivoTag 680 XL without transferrin
Group 2—human IgG-loaded SLNPs labeled with VivoTag 680 XL with transferrin
Group 3—human IgG labeled with VivoTag 680 XL
Group 4—untreated control (injected with vehicle)

Animals were injected intravenously with SLNPs loaded with antibody or free antibody and anesthetized using isoflurane at 2.0-2.5% in a XGI-8 gas anesthesia system (Perkin Elmer). Once anesthetized, the animals were placed inside the imaging chamber. Fluorescence images were taken using the Living Image® version 4.5.1 software. Each image was acquired using four different fluorescence filter combinations: excitation (ex.) 600 nm/emission (em.) 710 nm, ex. 620 nm/em. 710 nm, ex. 640 nm/em. 710 nm and ex. 660 nm/em. 710 nm. The corresponding settings in the software were set to: Exposure: auto; binning: 8; Fstop: 2; FOV: D. Data analysis was performed using the Living Image® version 4.5.1 software. The filter set used for data analysis was ex. 660 nm/em. 710 nm.

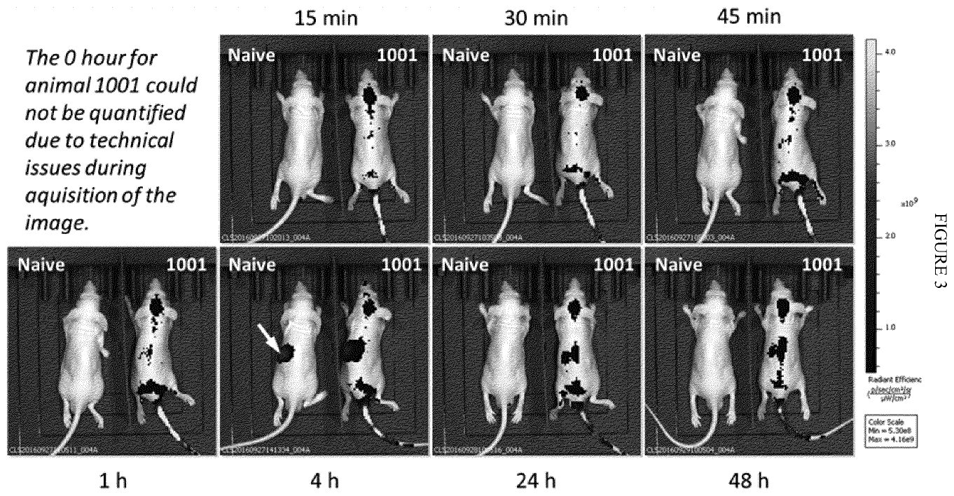

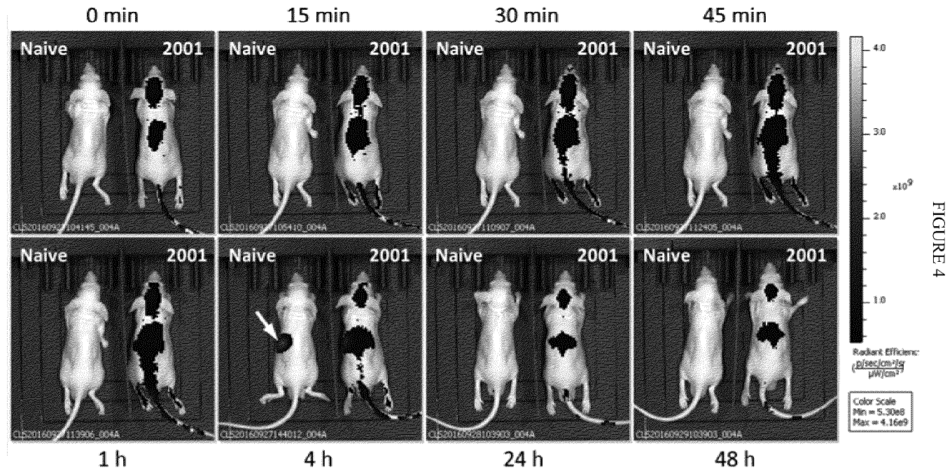

A representative result for the biodistribution of non-targeted SLNPs is given in FIG. 3. Only relatively small amounts of fluorescence-labeled particles were detected in the body and head region. Targeted SLNPs showed a more pronounced accumulation in head and mid-body as shown in FIG. 4. This reflected a distribution as would be expected from nanoparticles which are targeted via transferrin since liver and brain are those organs with the highest expression of transferrin receptor. The fluorescence in the naïve mouse observed after 4 h, marked with an arrow in the images, resulted from transfer of material during wake phases due to group housing with the dosed animal.

### V. In Vivo Distribution of SLNPs in Mouse Brain

Animals from in vivo imaging studies as described above were analyzed for transport of SLNPs across the blood-brain-barrier. Animals were injected with nanoparticles encapsulating human IgG or free human IgG as described above and, after 24 h, were sacrificed and perfused. The brain was extracted, fixed, sectioned and stained for the presence of human IgG or human serum albumin by immunohistochemistry.

The extraction as well as the immunohistochemical examination of the brain tissue was performed as follows. Animals from in vivo imaging studies were sacrificed after 24 h and perfused with phosphate buffered saline (PBS). Brains were surgically extracted and postfixed in 10% formalin at RT. Fixed brain tissue was dehydrated, freed from lipids and embedded in paraffin by following the incubation steps listed in Table 2.

TABLE 2
Fixation, dehydration, lipid removal and embedding
scheme for preparation of tissue sections.

| step | reagent | duration [min] |
|---|---|---|
| 1 | 10% formalin | 5 |
| 2 | 50% ethanol | 30 |
| 3 | 70% ethanol | 30 |
| 4 | 80% ethanol | 30 |
| 5 | 95% ethanol | 30 |
| 6 | 95% ethanol | 30 |
| 7 | 100% ethanol | 30 |
| 8 | 100% ethanol | 30 |
| 9 | xylol | 30 |
| 10 | xylol | 30 |
| 11 | melted paraplast | 25 |
| 12 | melted paraplast | 25 |
| 13 | melted paraplast | 25 |
| 14 | melted paraplast | 25 |

After solidification of paraffin, the embedded tissue was cut into slices of 5 μm thickness using a microtome. Tissue slices were transferred to microscope slides. Samples were subjected to deparaffination by following the incubation steps listed in Table 3.

TABLE 3
Deparaffination scheme for tissue slices
prior to (immuno-)histochemistry.

| step | reagent | duration [min] |
|---|---|---|
| 1 | xylol | 10 |
| 2 | xylol | 10 |
| 3 | 100% ethanol | 2 |
| 4 | 100% ethanol | 2 |
| 5 | 96% ethanol | 2 |
| 6 | 80% ethanol | 2 |
| 7 | 70% ethanol | 2 |
| 8 | water | as required |

Endogeneous peroxidase activity was blocked by incubation of samples in methanol: 30% hydrogen peroxide:water (7:1:2 volume ratio) for 10 min. Unspecific protein binding was prevented prior to immunostaining by a 20 min incubation in DAKO® protein block serum-free solution (DAKO Corporation).

Human IgG was specifically stained by immunohistochemistry using a rabbit antihuman IgG antibody (Abcam, ref. #: ab218427) at 1:200 fold dilution overnight at 4° C. A biotinylated secondary donkey anti-rabbit mAb (Jackson Immunoresearch, ref. #: 711-065-152) was used to detect the primary antibody for 2 h at RT. Biotinylated horseradish peroxidase was preincubated with avidin to form avidin-biotin-enzyme complexes. These complexes were transferred to the antibody-treated tissue slices for binding to biotinylated secondary antibodies. Detection of antigen was performed by adding hydrogen peroxide and 3,3′-diaminobenzidine (DAB) for 8 min at RT which are converted to a brown precipitate by horseradish peroxidase. Human serum albumin was detected analogously using the same method as described above. Here, a mouse anti-HSA monoclonal antibody (Abcam, ref. #: ab117455) was used as primary antibody. A biotinylated donkey anti-mouse serum (Jackson Immunoresearch, ref. #: 715-065-151) was used as secondary antibodies in a 1:500 dilution.

Samples were washed in water for 5 min after immunodetection and counterstained with eosin and hematoxylin according to common procedures. The samples were dehydrated by following the incubation steps given in Table 4 and mounted under coverslips using PERTEX® medium (Histolab).

TABLE 4
Dehydration scheme prior to mounting of
samples for light microscopy.

| step | reagent | duration |
|---|---|---|
| 1 | 70% ethanol | briefly immerse |
| 2 | 80% ethanol | |
| 3 | 96% ethanol | |
| 4 | 100% ethanol | |
| 5 | xylol | |

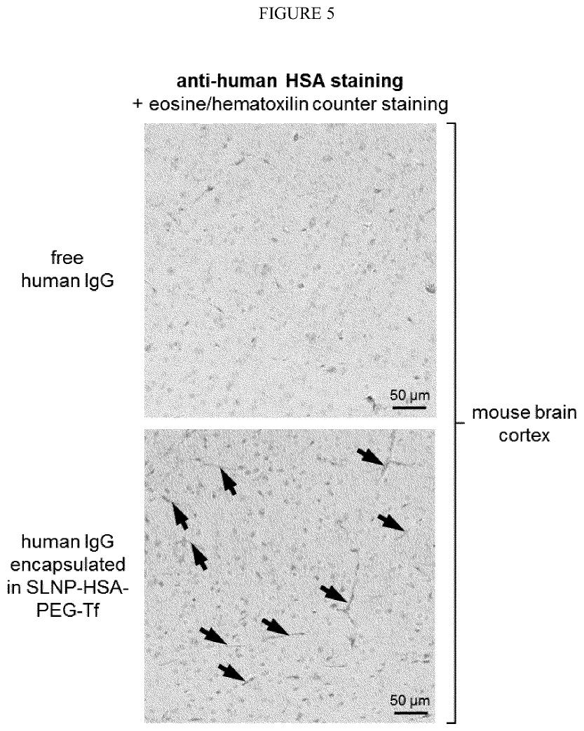

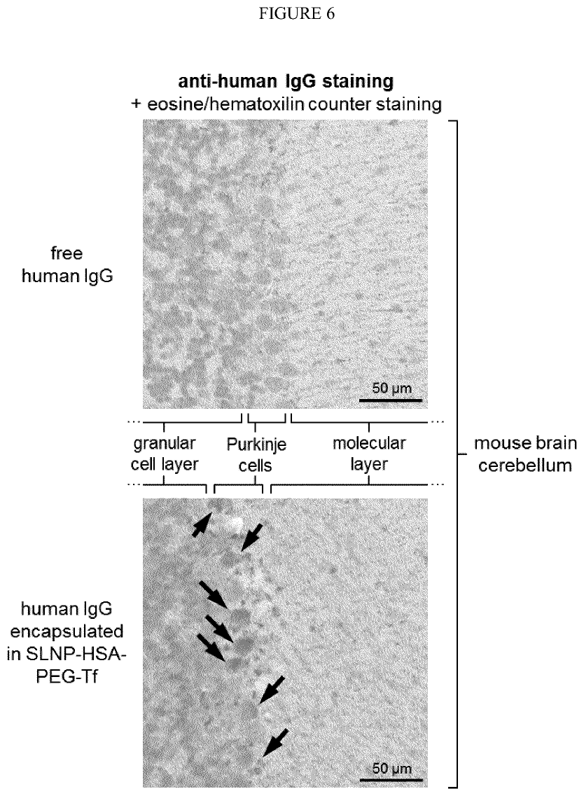

The stained tissue samples were analyzed and imaged using a light microscope. The results are depicted in FIGS. 5 and 6.

As can be seen from the images of FIG. 5, brain cortex endothelium of the mouse dosed with human IgG-loaded SLNP-HSA-PEG-Tf showed human HSA-specific staining (arrows), while brain cortex endothelium of the animal treated with human IgG solution did not. This indicates that human IgG-loaded SLNP-HSA-PEG-Tf was recruited by, and potentially transported to and across, the brain vascular endothelium, which is the major component of the blood-brain-barrier.

FIG. 6 shows that the brain cerebellum of the mouse dosed with human IgG-loaded SLNP-HSA-PEG-Tf showed human IgG-specific staining in the vicinity of Purkinje cells (arrows). In contrast, there was no human IgG-specific staining behind the blood-brain barrier in the brain cerebellum of the mouse treated with human IgG solution. Purkinje cells are known to highly express transferrin receptor (see for example Dickinson et al., Brain Res., 1998, Vol. 801(1-2); pp. 171-181) and are therefore expected to be capable of binding transferrin-targeted nanoparticles. The results shown in FIG. 6 indicate that human IgG-loaded SLNP-HSA-PEG-Tf that is equipped with an albumin corona carrying transferrin as a targeting ligand was transported into the brain and thus functioned as a carrier that transported its cargo (human IgG) over the blood-brain barrier.

This invention was made with the assistance of financial support from the Innovative Medical Initiative (IMI) under Grant Agreement No. 115363 (Collaboration on the Optimisation of Macromolecular Pharmaceutical Access to Cellular Targets—COMPACT).

1. A cargo substance-loaded nanoparticle modified with albumin and a targeting ligand, comprising
(i) a cargo substance selected from the group consisting of pharmaceutically active agents, cosmetically active agents and nutritional supplements;
(ii) a material which surrounds or embeds the cargo substance;
(iii) an albumin which is covalently directly or indirectly bound to the material (ii); and
(iv) a targeting ligand which is covalently bound to the albumin (iii) via a linker.

2. The nanoparticle as claimed in claim 1, where the cargo substance is a pharmaceutically active agent, especially a biopharmaceutical.

3. The nanoparticle as claimed in claim 1, where the nanoparticle is selected from the group consisting of
nanocapsules comprising a shell and a core, where the core comprises the cargo substance and the shell comprises the material (ii);
matrix particles containing the material (ii) in form of a matrix in which the cargo substance is embedded; and
mixed forms thereof.

4. The nanoparticle as claimed in claim 1, the where the material (ii) which surrounds or embeds the cargo substance is selected from the group consisting of lipids, natural polymers, synthetic polymers and carbon nanotubes.

5. The nanoparticle as claimed in claim 4, where
the lipids have a melting point of at least 25° C.;
the natural polymers are selected from the group consisting of polysaccharides, in particular starch, cellulose, pullulan or dextran; polyaminosaccharides, in particular chitosan, and polypeptides; and
the synthetic polymers are selected from the group consisting of poly(meth)acrylates, polystyrenes, polyethylene glycols, polyethyleneimines and polyesters of hydroxycarboxylic acids.

6. The nanoparticle as claimed in claim 4, where the lipids are selected from the group consisting of triglycerides, diglycerides, monoglycerides, fatty acids, steroids, and waxes.

7-8. (canceled)

9. The nanoparticle as claimed in claim 1, where the albumin which is covalently bound to the material (ii) is serum albumin, in particular human serum albumin, bovine serum albumin, monkey serum albumin, dog serum albumin, rat serum albumin or mouse serum albumin, specifically human serum albumin.

10. The nanoparticle as claimed in claim 1, where the targeting ligand is a ligand targeting cell surface proteins or lipids of the plasma membrane; in particular targeting receptors, ion channels or ganglioside M1.

11. (canceled)

12. The nanoparticle as claimed in any of the preceding claims, where the targeting ligand is selected from the group consisting of vitamins, polyoxyalkylene-containing polymers, peptides, proteins and deoxyribonucleic acids.

13. The nanoparticle as claimed in claim 9, where
the vitamins are selected from the group consisting of folic acid, the corresponding folate anion and thiamin;
the polyoxyalkylene-containing polymers are selected from poloxamers, in particular Poloxamer 188 and Poloxamer 407; and polysorbates, in particular polysorbate 80;
the peptides are selected from the group consisting of
Angiopep-2 (TFFYGGSRGKRNNFKTEEY)
ApoB (3371-3409) (SSVIDALQYKLEGTTRLTRKRGLKLATALSLSNKFVEGS)
ApoE (159-167)₂ ((LRKLRKRLL)₂)
Peptide-22 (Ac-C(&)MPRLRGC(&)-NH₂)
transferrin receptor binding-peptides, e.g. THR (THRPPMWSPVWP-NH₂) and retro-enantio THR (pwvpswmpprht-NH₂)
CRT (C(&)RTIGPSVC(&))
Leptin30 (YQQILTSMPSRNVIQISNDLENLRDLLHVL)
RVG29 (YTIWMPENPRPGTPCDIFTNSRGKRASNG)
^{D}CDX (greirtgraerwsekf)
Apamin (C(&1)NC(&2)KAPETALC(&1)ARRC(&2)QQH-NH₂)
MiniAp-4 ([Dap](&)KAPETALD(&))
reduced glutathione (GSH; gamma-L-glutamyl-L-cysteinylglycine)
G23 (HLNILSTLWKYRC)
G7 (GFtGFLS(O-beta-Glc)-NH₂)
TGN (TGNYKALHPHNG)
TAT (47-57) (YGRKKRRQRRR-NH₂)
SynB1 (RGGRLSYSRRRFSTSTGR)
diketopiperazines (&(N-MePhe)-(N-MePhe)Diketopiperazines)
PhPro ((Phenylproline)₄-NH₂)
EPRNEEK (EPRNEEK)
chlorotoxin (MC(&1)MPC(&2)FTTDHQMARKC(&3)DDC(&1) C(&4)GGKGRGKC(&2)YGPQC(&3)LC(&4)R—NH₂)
insulin (e.g., amino acid sequence set forth in GenBank accession no. V00565.1); and
peptides derived from tetanus toxin; and
the proteins are selected from the group consisting of
transferrin (e.g., as encoded by the polynucleotide sequence set forth in GenBank accession no. M12530.1 (mRNA) or AY308797.1 (genomic DNA))
apolipoprotein E3 (ApoE3) (e.g., as encoded by the polynucleotide sequence set forth in GenBank accession no. FJ525876.1 (DNA))
apolipoprotein A1 (ApoA1) (e.g., as encoded by the polynucleotide sequence set forth in GenBank accession no. J00098.1 (DNA))
apolipoprotein B100 (ApoB100) (e.g., as encoded by the polynucleotide sequence set forth in GenBank accession no. AH003569.2 (DNA))
antigen-binding molecules; in particular antibodies, antigen-binding fragments thereof, molecules comprising at least one antigen-binding region of an antibody, or antibody mimetics
tetanus toxin (e.g., amino acid sequence set forth in GenBank accession no. X04436.1)
CRM197 (non-toxic analog of the diphteria toxin, e.g., amino acid sequence set forth in GenBank accession no. X00703.1)
rabies virus glycoprotein (transmembrane glycoprotein G, e.g., amino acid sequence set forth in Genbank M13215.1)
the deoxyribonucleic acids are selected from
aptamers targeting a cell surface protein or a lipid of the plasma membrane.

14. (canceled)

15. The nanoparticle as claimed in claim 1, where the linker via which the targeting ligand is covalently bound to the albumin (iii) contains one or more polyalkyleneoxide chains, in particular one or more polyethyleneglycol chains, where the polyalkyleneoxide chains contain an overall amount of alkylene oxide repeating units of from 10 to 500, in particular of from 20 to 200.

16. (canceled)

17. A method for producing a nanoparticle as defined in any of the preceding claims, which method comprises
(a) providing a nanoparticle in which a cargo substance (i) is surrounded by or embedded in the material (ii);
(b) if necessary, modifying the material (ii) of the nanoparticle of step (a) in such a way that it can covalently bind the albumin (iii) either directly or via a linking group A;
(c) covalently attaching to the optionally modified nanoparticle
(c.1) the albumin; or
(c.2) the linking group A via which the albumin is to be attached to the optionally modified nanoparticle; or
(c.3) the linking group A to which the albumin is already attached; or
(c.4) the albumin which carries the covalently bound linker via which the targeting ligand is to be bound, or a part of the linker; or
(c.5) the albumin which carries the covalently bound linker to which the targeting ligand is attached; or
(c.6) the linking group A to which the albumin is already attached, where the albumin carries moreover the covalently bound linker via which the targeting ligand is to be bound, or a part of the linker; or
(c.7) the linking group A to which the albumin is already attached, where the albumin carries moreover the covalently bound linker to which the targeting ligand is attached;
(d.1) in case that step (c) is step (c.2), attaching to the linking group A of the product obtained in step (c.2)
(d.1.1) the albumin; or
(d.1.2) the albumin which carries the covalently bound linker via which the targeting ligand is to be bound, or a part of the linker; or
(d.1.3) the albumin which carries the covalently bound linker to which the targeting ligand is attached;
(d.2) in case that step (c) is step (c.1) or (c.3) and in case that step (d.1) is step (d.1.1), attaching to the albumin of the product obtained in step (c.1), (c.3) or (d.1.1)
(d.2.1) the linker or a part thereof; if necessary by reacting the albumin first with a linking group B and then with the linker or a part thereof; or
(d.2.2) the linker which already carries the targeting ligand; if necessary by reacting the albumin first with a linking group B and then with the linker already carrying the targeting ligand;
(e.1) in case that step (c) is step (c.4) or (c.6) and in case that step (d.1) is step (d.1.2) and in case that step (d.2) is step (d.2.1), for the case that only a part of the linker is contained in the product obtained in step (c.4), (c.6) (d.1.2) or (d.2.1), either
(e.1.1) converting the part of the linker into the complete linker; or
(e.1.2) reacting the part of the linker with the rest of the linker to which the targeting ligand is already attached; and
(e.2) in case that step (c) step is (c.4) or (c.6) and in case that step (d.1) is step (d.1.2) and in case that step (d.2) is step (d.2.1), for the case that the complete linker is contained in the product obtained in step (c.4), (c.6) (d.1.2) or (d.2.1), and in case that step (e.1) is step (e.1.1), attaching the targeting ligand to the linker.

18. The method as claimed in claim 1, where the material (ii) is a lipid and the cargo substance is stable in water; and where for providing in step (a) a nanoparticle in which the cargo substance (i) is surrounded by or embedded in the material (ii) and modifying the material (ii) of the nanoparticle in such a way that it can covalently bind the albumin (iii),
(a.1) the lipid, a functionalized lipid and one or more surfactants are dissolved in an organic solvent;
(a.2) the solution obtained in step (a.1) is mixed with a solution of the cargo substance in water to give a water-in-oil emulsion; and
(a.3) the water-in-oil emulsion obtained in step (a.2) is transferred to an aqueous phase to give a water-in-oil-in-water double emulsion.

19-22. (canceled)

23. The method as claimed in claim 12, where the solution of the cargo substance in water contains the cargo substance in an overall amount of up to 200 g per 1 of the solution.

24. The method as claimed in claim 12, where the weight ratio of the water-in-oil emulsion obtained in step (a.2) and the aqueous phase to which the former is transferred in step (a.3) is of from 1:10 to 1:1000.

25. The method as claimed in claim 14, where the water-in-oil emulsion obtained in step (a.2) is transferred in step (a.3) to the aqueous phase via an orifice, in particular via a syringe needle, of a diameter of at most 1400 μm.

26. (canceled)

27. A pharmaceutical composition containing a plurality of nanoparticles as claimed in any of claims 1 to 16 and a pharmaceutically acceptable carrier.

28. Nanoparticles as claimed in any of claims 1 to 16, for use as a medicament.

29. (canceled)

30. A method for producing nanoparticles in which a cargo substance which is stable in aqueous solution is embedded in or surrounded by a lipid material comprising
(1) dissolving in an organic solvent the lipid material, one or more surfactants and optionally one or more substances which under the given conditions are suitable to provide the lipid material with anchoring groups for further reactions;
(2) mixing the solution obtained in step (1) with a solution of the cargo substance in water to give a water-in-oil emulsion; and
(3) transferring the water-in-oil emulsion obtained in step (2) to an aqueous phase to give an oil-in-water emulsion.


SEQUENCE LISTING (19 sequences)

SEQ ID NO: 1 - protein, 25 aa, Artificial Sequence
  LDL receptor-binding domain of ApoE4
YLRVRLASHLRKLRKRLLRDADDLY

SEQ ID NO: 2 - protein, 29 aa, Artificial Sequence
  Acetylcholine receptor-binding domain of RVG (RVG29)
YTIWMPENPRPGTPCDIFTNSRGKRASNG

SEQ ID NO: 3 - protein, 19 aa, Artificial Sequence
  Angiopep-2
TFFYGGSRGKRNNFKTEEY

SEQ ID NO: 4 - protein, 39 aa, Artificial Sequence
  ApoB (3371-3409)
SSVIDALQYKLEGTTRLTRKRGLKLATALSLSNKFVEGS

SEQ ID NO: 5 - protein, 18 aa, Artificial Sequence
  ApoE (159-167)2
LRKLRKRLLLRKLRKRLL

SEQ ID NO: 6 - protein, 8 aa, Artificial Sequence
  MOD_RES 1: ACETYLATION
  DISULFID join(1,8)
  Peptide-22
  MOD_RES 8: AMIDATION
CMPRLRGC

SEQ ID NO: 7 - protein, 12 aa, Artificial Sequence
  transferrin receptor binding-peptide THR
  MOD_RES 12: AMIDATION
THRPPMWSPVWP

SEQ ID NO: 8 - protein, 9 aa, Artificial Sequence
  DISULFID join(1,9)
  CRT
CRTIGPSVC

SEQ ID NO: 9 - protein, 30 aa, Artificial Sequence
  Leptin30
YQQILTSMPSRNVIQISNDLENLRDLLHVL

SEQ ID NO: 10 - protein, 17 aa, Artificial Sequence
  DISULFID join(1,10)
  Apamin
  DISULFID join(2,14)
  MOD_RES 17: AMIDATION
CCKAPETALCARRCQQH

SEQ ID NO: 11 - protein, 8 aa, Artificial Sequence
  MOD_RES 1: N- and C-terminus linked by diaminopropyl
  MiniAp-4
KAPETALD

SEQ ID NO: 12 - protein, 13 aa, Artificial Sequence
  G23
HLNILSTLWKYRC

SEQ ID NO: 13 - protein, 7 aa, Artificial Sequence
  G7
  MOD_RES 3: Thr is D amino acid
  CARBOHYD 7: O-linked beta-Glc
  MOD_RES 7: AMIDATION
GFTGFLS

SEQ ID NO: 14 - protein, 12 aa, Artificial Sequence
  TGN
TGNYKALHPHNG

SEQ ID NO: 15 - protein, 11 aa, Artificial Sequence
  TAT (47-57)
  MOD_RES 11: AMIDATION
YGRKKRRQRRR

SEQ ID NO: 16 - protein, 18 aa, Artificial Sequence
  SynB1
RGGRLSYSRRRFSTSTGR

SEQ ID NO: 17 - protein, 4 aa, Artificial Sequence
  MOD_RES 1..4: phenylated
  PhPro
  MOD_RES 4: AMIDATION
PPPP

SEQ ID NO: 18 - protein, 7 aa, Artificial Sequence
  EPRNEEK
EPRNEEK

SEQ ID NO: 19 - protein, 36 aa, Leiurus quinquestriatus
  chlorotoxin
  DISULFID join(2,19)
  DISULFID join(5,28)
  DISULFID join(15,33)
  DISULFID join(20,35)
  MOD_RES 36: AMIDATION
MCMPCFTTDHQMARKCDDCCGGKGRGKCYGPQCLCR